(19) United States
(12) Patent Application Publication
(10) Pub. No.: US 2010/0267029 A1
(43) Pub. Date: Oct. 21, 2010

(54) METHOD OF IDENTIFYING THE PLANT SPECIES OF THE GENUS UNCARIA

(75) Inventor: Hiroki Yamaji, Ibaraki (JP)

Correspondence Address: LUCAS & MERCANTI, LLP, 475 PARK AVENUE SOUTH, 15TH FLOOR, NEW YORK, NY, 10016, US

(73) Assignee: TSUMURA & CO., Tokyo (JP)

(21) Appl. No.: 12/670,097

(22) PCT Filed: Jul. 25, 2008
(86) PCT No.: PCT/JP2008/063820

§ 371 Date: Jan. 21, 2010

(30) Foreign Application Priority Data
Jul. 26, 2007 (JP) 2007-194973

(51) Int. Cl. C12Q 1/68; G01N 33/15

(52) U.S. Cl. 435/6; 436/94

(57) ABSTRACT

The present invention provides a method of identifying the plant species of the genus Uncaria with high accuracy. The present invention relates to a method of identifying the plant species of the genus Uncaria contained in a test sample, comprising comparing the nucleotide sequence of the ITS region of ribosomal DNA of the plant contained in a test sample with at least one ITS region nucleotide sequence selected from among the nucleotide sequences represented by SEQ ID NOS: 1 to 11.

### TECHNICAL FIELD

The present invention relates to a method of identifying the plant species of the genus Uncaria by a genetic method.

### BACKGROUND ART

Diao-teng-gou (also referred to as Uncaria Thorn) is an herbal medicine listed in the Supplement 1 of the Japanese Pharmacopoeia 14th ed. The following three species are original plants thereof: Gouteng (Uncaria rhynchophylla Miguel); Huagouteng (Uncaria sinensis Haviland); and Daxiegouteng (Uncaria macrophylla Wallich). However, herbal medicines derived from species other than such three species have been marketed because there are 11 plant species of the genus Uncaria that have been distributed throughout China (Hsue H. & Wu H., 1999. Uncaria in Lo H. ed. Flora Reipublicae Popularis Sinicae 71(1) Science Press, Beijing). It has been difficult to distinguish genuine herbal medicines consisting of any one of the three above species of the genus Uncaria from such other herbal medicines. Therefore, there has been a problem of quality stability of Diao-teng-gou as Chinese natural medicine formulations. Although a technique for identifying plants of the genus Uncaria distributed throughout China based on their chemical components or anatomical features has been reported (Sakakibara et al., (1999) The Journal of Japanese Botany 74: 42-52), it has been necessary to establish a method of identifying the plant species of the genus Uncaria with improved accuracy.

As an aside, recent developments in molecular genetics have significantly influenced taxonomic methodology. That is, in addition to conventional morphological classification, it has become possible to assume an evolutional process based on gene similarity by selecting specific genes and comparing the nucleotide sequences of organism species and classify.

Surprisingly, genetic differences among organism species are conserved in genes encoding proteins for regulating basic vital activities. However, such differences vary in many genes according to the distribution of functions of individual proteins. Thus, regarding some genes, it is difficult to identify whether they are derived from the same origin. In view of the above, various classifications can be carried out by selecting an appropriate gene region depending on the range of organism species to be classified. For example, it is possible to classify from a wide range of genetic affiliation such as the relationship between whales and other mammals to a limited range of genetic affiliation such as regional differences in crows. Many genes such as a ribosomal RNA gene (Bruce Alberts et al., Essential Cell Biology, Garland Publishing Inc., 439-440, 1998) and a mitochondrial gene (Satoshi Horai, Cell Technology, vol. 14, 1031-1035, 1995) have been reported as a generally most-used gene for evolutionary assumption of relative distance and classification in terms of evolution as described above.

In the case of eukaryotic ribosomal RNA, RNA transcribed by RNA polymerase is separated into several fragments via processing. The fragments are incorporated into a small subunit (SS: Small Subunit) and a large subunit (LS: Large Subunit), which constitute a ribosome. RNA incorporated into the small subunit is referred to as 18SrRNA and consists of approximately 1900 bases (yeast) to 2000 bases (human). There are two types of RNA incorporated into the large subunit, namely, 28SrRNA and 5.8SrRNA. 28SrRNA consists of approximately 4700 bases (yeast) to 5000 bases. 5.8SrRNA consists of approximately 160 bases (yeast or human).

DNAs encoding the above rRNAs are located on the rRNA gene culsterencoding ribosomal RNAs, in the order of 18S, 5.8S, and 28S. A non-coding region called ITS (Internal Transcribed Spacer) is located between 18S and 5.8S and between 5.8S and 28S. ITSs presented in ribosomal DNA cluster encoding the above ribosomal RNA are universally present in plants. Many plants have been reported to contain ITSs. However, there are no reports regarding nucleotide sequences of many plant species of the genus Uncuria, classification of the plants based on such nucleotide sequence, or identification of the species based on such classification.

### DISCLOSURE OF THE INVENTION

It is an object of the present invention to provide a method of identifying the plant species of the genus Uncaria with high accuracy.

As a result of intensive studies in order to achieve the above object, the present inventors have found that species-specific polymorphisms are present in the nucleotide sequences of ITS regions in ribosomal DNAs of plants belonging to the genus Uncaria, and that the plant species of the genus Uncaria can be identified by using such polymorphisms. This has led to the completion of the present invention.

Specifically, the present invention encompasses the following inventions.

(1) A method of identifying the plant species of the genus Uncaria contained in a test sample, comprising comparing the nucleotide sequence of the ITS region in plant ribosomal DNA contained in a test sample with the nucleotide sequence of at least one ITS region selected from among the nucleotide sequences represented by SEQ ID NOS: 1 to 11.
(2) The method according to (1), wherein the test sample is an herbal medicine.
(3) The method according to (1) or (2), comprising identifying whether the plant of the genus Uncaria contained in a test sample is a species selected from the group consisting of Uncaria rhynchophylla, Uncaria sinensis, Uncaria macrophylla, Uncaria wangii, Uncaria scandens, Uncaria homomella, Uncaria hirsuta, Uncaria lancifolia, Uncaria sessilifructus, Uncaria laevigata and Uncaria yunnanensis.
(4) The method according to (1) or (2), comprising identifying whether or not the plant of the genus Uncaria contained in a test sample is a species selected from the group consisting of Uncaria rhynchophylla, Uncaria sinensis and Uncaria macrophylla.
(5) The method according to (4), comprising comparing at least one base of the nucleotide sequence of the ITS region in plant ribosomal DNA contained in a test sample, selected from among bases located at positions corresponding to positions 12, 37, 43, 47, 78, 79, 84, 85, 86, 94, 104, 105, 169, 172, 177, 209, 210, 211, 215, 365, 395, 412, 413, 415, 433, 446, 447, 450, 451, 461, 514, 518, 526, 532, 537, 546, 554, 567, 570, 585, 588, 590, 592, 608 and 609 of the nucleotide sequence represented by SEQ ID NO: 1 used as a reference sequence with the selected nucleotide sequence of SEQ ID NO: 1.
(6) The method according to (5), comprising comparing at least one base of the nucleotide sequence of the ITS region in plant ribosomal DNA contained in a test sample, selected from among bases located at positions corresponding to positions 12, 37, 43, 47, 79, 84, 85, 86, 94, 104, 105, 169, 172, 177, 209, 210, 211, 215, 365, 395, 412, 413, 415, 433, 446, 447, 450, 451, 461, 514, 518, 526, 532, 537, 546, 554, 567, 570, 585, 588, 590, 592, 608 and 609 of the nucleotide sequence represented by SEQ ID NO: 1 used as a reference sequence with the selected nucleotide sequence of SEQ ID NO: 1.
(7) The method according to (6), comprising comparing at least one base of the nucleotide sequence of the ITS region in plant ribosomal DNA contained in a test sample, selected from among bases located at positions corresponding to positions 12, 37, 43, 85, 94, 104, 169, 177, 209, 211, 215, 412, 413, 415, 450, 461, 532, 537, 585, 588 and 608 of the nucleotide sequence represented by SEQ ID NO: 1 used as a reference sequence with the selected nucleotide sequence of SEQ ID NO: 1.

The present invention provides a method of identifying the plant species of the genus Uncaria with high accuracy.

### BRIEF DESCRIPTION OF THE DRAWINGS

FIG. 1 shows detailed information on 64 mutation sites found as a result of examination via alignment of the nucleotide sequences of the ITS regions of ribosomal DNAs determined for a plurality of samples derived from 11 species of the genus Uncaria.

FIG. 2-1 shows results of alignment of the nucleotide sequences of the ITS regions obtained from samples derived from 42 species listed in Table 3.

FIG. 2-2 shows results of alignment of the nucleotide sequences of the ITS regions obtained from samples derived from 42 species listed in Table 3.

FIG. 2-3 shows results of alignment of the nucleotide sequences of the ITS regions obtained from samples derived from 42 species listed in Table 3.

FIG. 2-4 shows results of alignment of the nucleotide sequences of the ITS regions obtained from samples derived from 42 species listed in Table 3.

FIG. 2-5 shows results of alignment of the nucleotide sequences of the ITS regions obtained from samples derived from 42 species listed in Table 3.

FIG. 2-6 shows results of alignment of the nucleotide sequences of the ITS regions obtained from samples derived from 42 species listed in Table 3.

FIG. 2-7 shows results of alignment of the nucleotide sequences of the ITS regions obtained from samples derived from 42 species listed in Table 3.

FIG. 2-8 shows results of alignment of the nucleotide sequences of the ITS regions obtained from samples derived from 42 species listed in Table 3.

FIG. 2-9 shows results of alignment of the nucleotide sequences of the ITS regions obtained from samples derived from 42 species listed in Table 3.

FIG. 2-10 shows results of alignment of the nucleotide sequences of the ITS regions obtained from samples derived from 42 species listed in Table 3.

FIG. 2-11 shows results of aligmnent of the nucleotide sequences of the ITS regions obtained from samples derived from 42 species listed in Table 3.

The disclosure of Japanese Patent Application No. 2007-194973, which is a priority document of the present application, is incorporated herein by reference.

### DESCRIPTION OF THE PREFERRED EMBODIMENTS

It has been known that ribosomal RNA is involved in protein synthesis, which is essential for living activities of all organisms, that most organisms contain a plurality of copies of ribosomal DNA (rDNA) encoding ribosomal RNA, and that eukaryotes contain several hundreds to several tens of thousands of copies thereof. In the eukaryotes, rDNA generally contains regions encoding 18SrRNA, 5.8SrRNA and 28SrRNA (referred to as 18SrDNA, 5.8SrDNA and 28SrDNA, respectively) in series, and constructs repetitive sequences. ITS1 (Internal Transcribed Spacer 1) exists between 18S and 5.8S and ITS2 exists between 5.8S and 28S. Herein, the two above ITSs and 5.8S existing therebetween are collectively referred to as an “ITS region.”

Ribosomal DNA (rDNA) sequences are widely used for organism classification. This is because such sequences are highly conserved in organisms from the simplest eukaryotes such as yeasts to highly complicated eukaryotes such as mammals. However, it can be expected that a certain amount of mutation is caused in a partial sequence that is discarded during processing, such as ITS. As the present invention, in order to examine differences among hybrid species, certain amount of differences between sequences is necessary. In contrast, when excessive differences are present in sequences, problems would arise in sequencing or in amplified primers in some cases. With regard to sequencing of ITS and surround thereof, ribosomal DNA allows searching for highly dispersed ITSs with the use of highly conserved SS and LS regions.

The plants of the genus Uncaria include, for example, Uncaria rhynchophylla, Uncaria sinensis, Uncaria macrophylla, Uncaria wangii, Uncaria scandens, Uncaria homomella, Uncaria hirsuta, Uncaria luncifvlia, Uncaria sessilifructus, Uncaria laevigata, Uncaria yunnanensis, Uncaria gambir, Uncaria guianensis, and Uncaria tomentosa.

The present inventors determined the nucleotide sequences (SEQ ID NOS: 12 to 53) of ITS regions (each comprising ITS1, ITS2, and 5.8S existing therebetween) contained in ribosomal DNAs of a plurality of samples derived form the following species (11 species in total): three original plant species of an herbal medicine Diao-teng-gou listed on the Japanese Pharmacopoeia: Uncaria rhynchophylla (Gouteng: Uncaria rhynchophylla Miguel), Uncaria sinensis (Huagouteng: Uncaria sinensis Haviland), and Uncaria macrophylla (Daxiegouteng: Uncaria macrophylla Wallich); and 8 other plant species of the genus Uncaria distributed throughout China: Uncaria wangii, Uncaria scandens, Uncaria homomella, Uncaria hirsuta, Uncaria lancifoliu, Uncaria sessilifructus, Uncaria laevigata, and Uncaria yunnanensis. From the result of comparison of the aligned sequences, species-specific polymorphisms are found in the nucleotide sequences of ITS regions in ribosomal DNAs of plants of the genus Uncaria.

Specifically, the present inventors have found that the plant species of the genus Uncuria can be identified by comparing the nucleotide sequence of an ITS region in plant ribosomal DNA contained in a test sample with the nucleotide sequence of at least one ITS region selected from among the nucleotide sequences represented by SEQ ID NOS: 1 to 11. SEQ ID NO: 1 represents the nucleotide sequence of an ITS region of Uncaria rhynchophylla. SEQ ID NO: 2 represents the nucleotide sequence of an ITS region of Uncaria sinensis. SEQ ID NO: 3 represents the nucleotide sequence of an ITS region of Uncaria wangii. SEQ ID NO: 4 represents the nucleotide sequence of an ITS region of Uncaria scandens. SEQ ID NO: 5 represents the nucleotide sequence of an ITS region of Uncaria homomella. SEQ ID NO: 6 represents the nucleotide sequence of an ITS region of Uncaria hirsute. SEQ ID NO: 7 represents the nucleotide sequence of an ITS region of Uncaria lancifolia. SEQ ID NO: 8 represents the nucleotide sequence of an ITS region of Uncaria sessilifructus. SEQ ID NO: 9 represents the nucleotide sequence of an ITS region of Uncaria laevigata. SEQ ID NO: 10 represents the nucleotide sequence of an ITS region of Uncaria macrophylla. SEQ ID NO: 11 represents the nucleotide sequence of an ITS region of Uncaria yunnanensis.

According to the method of the present invention, at least the 11 above plant species of the genus Uncaria can be identified. The method of the present invention is useful for identification of three species in particular, that is to say, Uncaria rhynchophylla, Uncaria sinensis and Uncaria macrophylla.

In the present invention, identification of the plant species of the genus Uncaria encompasses identification of whether or not the species is a specific species, or of whether or not the species is included in a specific group of species.

For instance, in one embodiment of the method of the present invention, it is possible to identify whether or not a plant of the genus Uncaria contained in a test sample corresponds to any one of the three species (i.e., Uncaria rhynchophylla, Uncaria sinensis, and Uncaria macrophylla) described as original plants of the herbal medicine Diao-teng-gou listed in the Japanese Pharmacopoeia. In other words, it is possible to identify whether or not a test sample is a genuine sample of the herbal medicine Diao-teng-gnu listed in the Japanese Pharmacopoeia.

Regarding an identification method comprising comparing the nucleotide sequence of an ITS region in ribosomal DNA of a plant contained in a test sample with at least one ITS region nucleotide sequence selected from among nucleotide sequences represented by SEQ ID NOS: 1 to 11, any one of the following methods can be used: a method comprising comparing the entire nucleotide sequence of an ITS region; a method comprising comparing bases located at one to several specific sites; and a method comprising combining a plurality of base differences at one to several specific sites and comparing the resulting combination patterns.

An example of a method comprising comparing the entire nucleotide sequence of an ITS region is a method comprising determining the nucleotide sequence of an ITS region in ribosomal DNA of a plant contained in a test sample, comparing the nucleotide sequence with the nucleotide sequence represented by SEQ ID NOS: 1 to 11, and identifying the species based on homology percentage. In general, even among closely related organism species, slight homology differences can be observed, reflecting the evolutional process. Therefore, in many cases, the nucleotide sequence of ribosomal DNA obtained from a test sample would not necessarily be 100% identical to the nucleotide sequence represented by any one of SEQ ID NOS: 1 to 11. In such case, it is also possible to readily assume a relatively close relationship by obtaining homology percentage.

An example of a method of determining the nucleotide sequence of an ITS region is a method of determining a nucleotide sequence by extracting DNA from a test sample, amplifying an ITS region with the use of the extracted DNA as a template, and carrying out direct sequencing or cloning. Amplification of an ITS region, direct sequencing, cloning, and determination of the nucleotide sequence can be carried out by conventional methods. For example, in addition to PCR, the LAMP method (Eiken Chemical Co., Ltd.), the ICAN method (Takara Shuzo Co., Ltd.), or the like can be used for ITS region amplification.

Accordingly, in one embodiment, the method of the present invention comprises: 1) extracting DNA from a test sample; 2) amplifying an ITS region with the use of the DNA as a template; 3) determining the nucleotide sequence of the ITS region; and 4) comparing the obtained nucleotide sequence with at least one nucleotide sequence represented by SEQ ID NOS: 1 to 11 to identify the plant species as the species of the genus Uncaria with the highest homology thereto.

For primers for amplifying an ITS region, known universal primers can be used (White T. J. et al., 1990, Amplification and direct sequencing of fungal ribosomal RNA genes for phylogenetics, In PCR Protocols, A Guide to Methods and Applications (M. A. Innis, D. H. Gelfand, J. J. Sninsky, and T. J. White, Eds.) pp. 315-322. Academic Press, San Diego).

Primers for amplifying an ITS region may be designed based on the nucleotide sequence of an ITS region in ribosomal DNA of a plant of the genus Uncaria. Conditions for designing of primers are not particularly limited. For instance, such primers may be obtained by designing a nucleotide which is a part of any one of or plurality of nucleotide sequences represented by SEQ ID NOS: 1 to 11 or of a nucleotide sequence(s) complementary thereto and which has a continuous nucleotide sequence comprising 10 or more bases. If a nucleotide sequence comprising fewer than 10 bases is used, it is statistically predicted that a plurality of nucleotide sequences complementary to the nucleotide sequence would exist on template DNA, resulting in reduced precision due to the primers used for amplification. Therefore it is preferable to use a nucleotide sequence comprising 10 or more bases. In addition, if a nucleotide sequence comprising 10 bases or more bases is used, a nucleotide sequence used as a primer, wherein 10% or less bases thereof have substitution, deletion, insertion, or addition, can hybridize with template DNA and thus may be used as a primer. Primers, for example, can be synthesized with the use of a commercially available DNA synthesizing apparatus or the like.

Specific examples of primers for amplifying an ITS region in ribosomal DNA of a plant of the genus Uncaria are described below.

As a primer for simultaneously amplifying ITS1, 5.8S, and ITS2 (i.e., ITS region), for example, the following primer sets may be used: ITS5 (5′-GGA AGT AAA AGT CGT AAC AAG G-3′) and ITS4 (5′-TCC TCC GCT TAT TGA TAT GC-3′) (White et al. 1990 described above). Herein, “ITS5” and “ITS4” are primer names.

As primers for separately amplifying ITS1 and ITS2, for example, the following primer sets may be used: ITS2 (5′-GCT GCG TTC TTC ATC GAT GC-3′) and ITS3 (5′-GCA TCG ATG AAG AAC GCA GC-3′) (White et al. 1990 described above). Herein, “ITS2” and “ITS3” are primer names.

Alternatively, ITS1 and ITS2 may be separately amplified as 2 fragments (4 fragments in total) by using the following primer set as primers designed for a conserved region in a plant of the genus Uncaria:

Amplification of ITS1: ITS5 & ITS-UncariaR2
(5′-GAG TTT TCC TTG GCG CGT TCC G-3′),
ITS-UncariaF1
(5′-CGC GCG GAA AAC GTA ACT CAA-3′) & ITS2;
and
Amplification of ITS2: ITS3 & ITS-UncariaR1
(5′-ATT CAA CCA CCA CTC GTC GTG-3′),
ITS-UncariaF2
(5′-CTA AAT GAG AGT CCT CGG CG-3′) & ITS4.

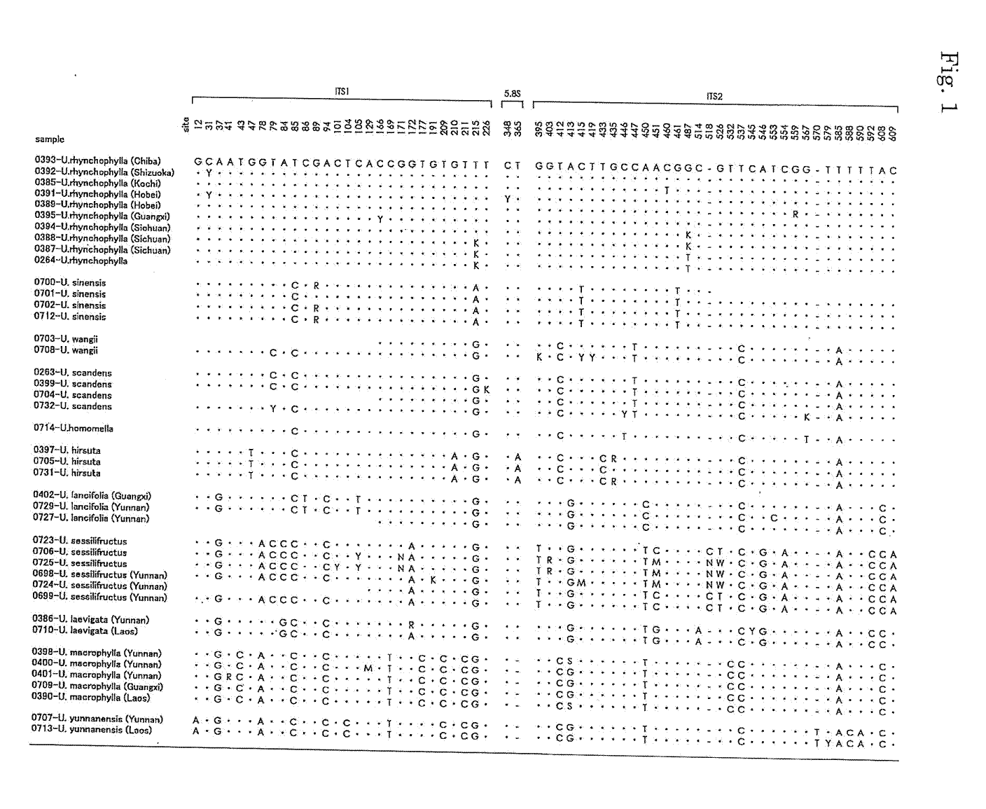

Further, the present inventors aligned nucleotide sequences of ITS regions in ribosomal DNAs determined for a plurality of samples of the 11 above species belonging to the genus Uncaria. As a result, 64 mutation sites have been found. In addition, they have found that the plant species of the genus Uncaria can be also identified by comparing only the bases located at specific sites in the ITS regions. FIG. 1 summarizes detailed information on bases at the above 64 mutation sites for the species and the samples. In the comparison of nucleotide sequences in the present invention, the species of the genus Uncaria can be identified by comparing differences in the 64 mutation sites with a method comprising comparing bases at one to several specific sites.

Specifically, in one embodiment of the present invention, the plant species of the genus Uncaria can be identified by comparing at least one, at least two, at least three, preferably one to ten, more preferably one to five, and further preferably one to three bases of the nucleotide sequence of the ITS region in plant ribosomal DNA contained in a test sample, selected from among bases located at positions corresponding to positions 12, 37, 43, 47, 78, 79, 84, 85, 86, 94, 104, 105, 169, 172, 177, 209, 210, 211, 215, 365, 395, 412, 413, 415, 433, 446, 447, 450, 451, 461, 514, 518, 526, 532, 537, 546, 554, 567, 570, 585, 588, 590, 592, 608, and 609 of the nucleotide sequence represented by SEQ ID NO: 1 used as a reference sequence with the selected nucleotide sequence of SEQ ID NO: 1.

Of these, preferably, at least one, at least two, at least three, preferably one to ten, more preferably one to five, and further preferably one to three bases of the nucleotide sequence of the ITS region in plant ribosomal DNA contained in a test sample, selected from among bases located at positions corresponding to positions 12, 37, 43, 47, 79, 84, 85, 86, 94, 104, 105, 169, 172, 177, 209, 210, 211, 215, 365, 395, 412, 413, 415, 433, 446, 447, 450, 451, 461, 514, 518, 526, 532, 537, 546, 554, 567, 570, 585, 588, 590, 592, 608, and 609 of the nucleotide sequence represented by SEQ ID NO: 1 used as a reference sequence are compared with a selected nucleotide sequence of SEQ ID NO: 1.

Herein, the phrase “bases located at positions corresponding to positions X . . . of the nucleotide sequence represented by SEQ ID NO: 1 used as a reference sequence” is used to define the position of a certain base in the nucleotide sequence of an arbitrary ITS region with the use of the nucleotide sequence represented by SEQ ID NO: 1 as a reference sequence. Specifically, when the nucleotide sequence of an ITS region derived from a test sample (hereinafter referred to as “nucleotide sequence Y”) is subjected to alignment with the nucleotide sequence represented by SEQ ID NO: 1, such phrase means a base in the nucleotide sequence Y that is aligned with a base at position X reckoned from the first base of the nucleotide sequence represented by SEQ ID NO: 1, (that is to say, when the bases are aligned on the same tandem array for alignment). In addition, when the nucleotide sequence of an ITS region derived from a test sample (hereinafter referred to as “nucleotide sequence Y”) is subjected to alignment with the nucleotide sequence represented by SEQ ID NO: 1 used as a reference sequence, the phrase “bases located at positions corresponding to positions V and W . . . of the nucleotide sequence represented by SEQ ID NO: 1 used as a reference sequence” means a base-inserted between two bases in the nucleotide sequence, which are aligned with bases at positions V and W reckoned from the first base in the nucleotide sequence represented by SEQ ID NO: 1. Alternatively, such expression is used to express the insertion or non-insertion of such base. When the position of a base is described as above with the use of the nucleotide sequence represented by SEQ ID NO: 1 as a reference sequence, a base at position 518 positioned next to T at position 517 of SEQ ID NO: 1 is deleted, and G positioned next to C at position 517 and A positioned next to G are located at positions 519 and 520, respectively. In addition, when bases are reckoned as described above, a base at position 570 positioned next to T at position 569 (base at position 568 of SEQ ID NO: 1) is deleted, and G positioned next to T at position 569 (base at position 569 of SEQ ID NO: 1) and A positioned next to G (base at position 570 of SEQ ID NO: 1) are located at positions 571 and 572, respectively. According to the present invention, the above positions of bases correspond to the positions of bases determined by alignment with the use of alignment software such as BioEdit (http://www.mbio.ncsu.edu/BioEdit/bioedit.html). BioEdit can be downloaded from the following website: http://www.mbio.ncsu.edu/BioEdit/BioEdit.zip.

The nucleotide sequence represented by SEQ ID NO: 1 corresponds to an ITS region comprising ITS1, 5.8S, and ITS2, provided that the position of the start base of ITS1 is designated as position 1. Herein, the sequence beginning with the start base of ITS1 of a plant belonging to the genus Uncaria is designated as 5′-TCGAATCCTG.

Alignment of the nucleotide sequence represented by SEQ ID NO: 1 with other nucleotide sequences may be manually carried out. Meanwhile, an alignment table may be prepared by using a multiple alignment program (Thompson, J. D. et al, (1994) Nucleic Acids Res. 22, pp. 4673-4680) for default setting. Such program can be obtained from the websites of, for example, the European Bioinformatics Institute (EBI: http://www.ebi.ac.uk/index.htinl) and the DNA Data Bank of Japan (DDBJ, http://www.ddbj.nig.ac.jp/Welcome-j.html) operated by the National Institute of Genetics. Persons skilled in the art can further adjust the obtained alignment results so as to achieve optimum alignment according to need. Preferably, such optimum alignment is performed in view of nucleotide sequence similarity, the frequency of gap insertion, etc.

More specifically, identification may be performed as described below. When a base at position 85 is T (thymine) and/or a base at position 215 is T or K (mixed nucleotide sequence: T and G (glycine)), the plant species can be identified as Uncaria rhynchophylla. When a base at position 215 is A (adenine), a base at position 415 is T, and/or a base at position 461 is T, the plant species can be identified as Uncaria sinensis. When a base at position 43 is C (cytosine), a base at position 177 is C, and/or a base at position 532 is C, the plant species can be identified as Uncaria macrophylla. If at least one base corresponds to the above, the plant species can be identified as a specific species. For instance, as long as a base at position 85 of a test sample is T with the use of the nucleotide sequence represented by SEQ ID NO: 1 as a reference sequence, it is possible that the sample contains Uncaria rhynchophylla without examining other bases. The same applies to other bases.

When a base at position 537 is T and/or a base at position 585 is T, the plant species can be identified as Uncaria rhynchophylla or Uncaria sinensis. If at least one base corresponds to the above, the plant species can be identified as Uncaria rhynchophylla or Uncaria sinensis.

When, a base at position 412 is T, and a base at position 37 is A, a base at position 94 is A, a base at position 413 is A, a base at position 450 is A and/or a base at position 608 is C, the plant species can be identified as Uncaria rhynchophylla or Uncaria sinensis. The plant species can be identified as Uncaria rhynchophylla or Uncaria sinensis if at least two bases correspond to the above, that is to say, as long as it can be confirmed that a base position 412 is T and a base at positions 37, 94, 413, 450 or 608 corresponds to the above.

When a base at position 169 is T, a base at position 209 is C and/or a base at position 211 is C, and further a base at position 12 is G, a base at position 104 is T, a base at position 588 is T and/or a base at position 590 is T, the plant species can be identified as Uncaria macrophylla. Also, the plant species can be identified as Uncaria macrophylla if at least two bases of these bases correspond to the above, that is to say, as long as it can be confirmed that a base at position 169, 209, or 211 corresponds to the above and that a base at position 12, 104, 588, or 590 corresponds to the above.

Therefore, it is possible to identify whether or not a plant of the genus Uncaria contained in a test sample corresponds to any one of Uncaria rhynchophylla, Uncaria sinensis and Uncaria macrophylla by comparing at least one, for example, at least two, at least three, preferably one to ten, more preferably one to five, and further preferably one to three (e.g., one or two) base(s) of the nucleotide sequence of the ITS region in plant ribosomal DNA contained in a test sample, selected from among bases located at positions corresponding to positions 12, 37, 43, 85, 94, 104, 169, 177, 209, 211, 215, 412, 413, 415, 450, 461, 532, 537, 585, 588, and 608 of the nucleotide sequence represented by SEQ ID NO: 1 used as a reference sequence with a selected nucleotide sequence of SEQ ID NO: 1.

In addition, comparison of the following sites allows identification of the species other than Uncaria rhynchophylla, Uncaria sinensis, and Uncaria macrophylla.

When a base at position 12 is A, a base at position 104 is C, T is inserted at position 570, a base at position 588 is C and/or a base at position 590 is A, the plant species can be identified as Uncaria yunnanensis. That is to say, as long as at least one base corresponds to the above, the plant species can be identified as Uncaria yunnanensis.

When a base at position 47 is T, a base at position 210 is A, a base at position 365 is A and/or a base at position 433 is C, the plant species can be identified as Uncaria hirsuta. That is to say, as long as at least one base corresponds to the above, the plant species can be identified as Uncaria hirsuta.

When a base at position 86 is T, a base at position 105 is T and/or a base at position 450 is C, the plant species can be identified as Uncaria lancifolia. That is to say, as long as at least one base corresponds to the above, the plant species can be identified as Uncaria lancifolia.

When a base at position 84 is C, a base at position 395 is T, a base at position 451 is C or M (mixed nucleotide sequence: A and C), a base is inserted at position 518, a base at position 526 is T or W (mixed nucleotide sequence: A and T), a base at position 554 is A and/or a base at position 609 is A, the plant species can be identified as Uncaria sessilifructus. That is to say, as long as at least one base corresponds to the above, the plant species can be identified as Uncaria sessilifructus.

When a base at position 84 is G, a base at position 451 is G and/or a base at position 514 is A, the plant species can be identified as Uncaria laevigata. That is to say, as long as at least one base corresponds to the above, the plant species can be identified as Uncaria laevigata.

When a base at position 446 is T and/or a base at position 567 is T, the plant species can be identified as Uncaria homomella. That is to say, as long as at least one base corresponds to the above, the plant species can be identified as Uncaria homomella.

In other words, when the nucleotide sequence represented by SEQ ID NO: 1 is used as a reference sequence, as long as any one of bases at positions 12, 47, 84, 86, 104, 105, 210, 365, 395, 433, 446, 450, 451, 514, 518, 526, 554, 567, 570, 588, 590, and 609 corresponds to the above, the plant species contained in a test sample may be identified as the species other than Uncaria rhynchophylla, Uncaria sinensis, and Uncaria macrophylla.

Further, when a base at position 79 is C, a base at position 172 is A, a base at position 395 is T, a base at position 447 is T, a base at position 546 is G and/or a base at position 592 is C, the plant species can be identified as the species other than Uncaria rhvnchophylla, Uncaria sinensis and Uncaria macrophylla, although it is impossible to identify which species corresponds thereto. That is to say, as long as at least one base corresponds to the above, the plant species can be identified as the species other than Uncaria rhynchophylla, Uncaria sinensis and Uncaria macrophylla.

Therefore, it is possible to identify whether or not the plant species contained in a test sample is Uncaria rhvnchophylla, Uncaria sinensis or Uncaria macrophylla by comparing at least one, for example, at least two, at least three, preferably one to ten, more preferably one to five, and further preferably one to three (e.g., one or two) base(s) of the nucleotide sequence of the ITS region in plant ribosomal DNA contained in a test sample, selected from among bases located at positions corresponding to positions 12, 37, 43, 47, 79, 84, 85, 86, 94, 104, 105, 169, 172, 177, 209, 210, 211, 215, 365, 395, 412, 413, 415, 433, 446, 447, 450, 451, 461, 514, 518, 526, 532, 537, 546, 554, 567, 570, 585, 588, 590, 592, 608, and 609 of the nucleotide sequence represented by SEQ ID NO: 1 used as a reference sequence with a selected nucleotide sequence of SEQ ID NO: 1.

In the following Table 1 (medicinal species) and Table 2 (non-medicinal species), sites on the nucleotide sequence of an ITS region in ribosomal DNA of a plant of the genus Uncaria, which are useful for species identification, are provided when the nucleotide sequence represented by SEQ ID NO: 1 is used as a reference sequence.

TABLE 1

| | | |
|---|---|---|
| T at position 85 | | U. rhynchophylla |
| T or K at position 215 | | U. rhynchophylla |
| A at position 215 | | U. sinensis |
| T at position 415 | | U. sinensis |
| T at position 461 | | U. sinensis |
| C at position 43 | | U. macrophylla |
| C at position 177 | | U. macrophylla |
| C at position 532 | | U. macrophylla |
| T at position 537 | | U. rhynchophylla or |
| | | U. sinensis |
| T at position 585 | | U. rhynchophylla or |
| | | U. sinensis |
| A at position 37 | T at position 412 | U. rhynchophylla or |
| A at position 94 | | U. sinensis |
| A at position 413 | | |
| A at position 450 | | |
| C at position 608 | | |
| T at position 169 | G at position 12 | U. macrophylla |
| C at position 209 | T at position 104 | |
| C at position 211 | T at position 588 | |
| | T at position 590 | |
| C at position 79 | | species other than |
| A at position 172 | | U. rhynchophylla, U. sinensis |
| T at position 395 | | and U. macrophylla |
| T at position 447 | | |
| G at position 546 | | |
| C at position 592 | | |

TABLE 2

| | |
|---|---|
| A at position 12 | U. yunnanensis |
| C at position 104 | |
| T inserted at position 570 | |
| C at position 588 | |
| A at position 590 | |
| T at position 47 | U. hirsuta |
| A at position 210 | |
| A at position 365 | |
| C at position 433 | |
| T at position 86 | U. lancifolia |
| T at position 105 | |
| C at position 405 | |
| C at position 84 | U. sessilifructus |
| T at position 395 | |
| C or M at position 451 | |
| Base inserted at position 518 | |
| T or W at position 526 | |
| A at position 554 | |
| A at position 609 | |
| G at position 84 | U. laevigata |
| G at position 451 | |
| A at position 514 | |
| T at position 446 | U. homomella |
| T at position 567 | |

In addition, when a base at position 78 is A, the plant species can be identified as Uncaria macrophylla, Uncaria yunnanensis or Uncaria sessilifructus.

Comparison of the above bases located at specific positions may be carried out by determining the nucleotide sequence of an ITS region in ribosomal DNA of a plant contained in a test sample and comparing bases located at specific positions in the obtained nucleotide sequence of an ITS region with each other. The method of determining the nucleotide sequence of an ITS region is described above. Specifically, the nucleotide sequence can be determined by extracting DNA from a test sample amplifying an ITS region with the use of the DNA as a template, and carrying out direct sequencing or cloning. The same applies to primers.

Accordingly, in one embodiment, the method of the present invention comprises: 1) extracting DNA from a test sample; 2) amplifying an ITS region with the use of the DNA as a template, 3) determining the nucleotide sequence of the ITS region and 4) comparing at least one base selected from bases located at positions corresponding to the above positions of the nucleotide sequence represented by SEQ ID NO: 1 used as a reference sequence with at least one nucleotide sequence represented by any one of SEQ ID NOS: 1 to 11.

As a method of comparing bases at one to several specific sites, a method involving the use of PCR primers may be used. The method comprises comparing nucleotide sequences, designing PCR primers for a region with base differences between sequences, and identifying which primers can amplify the region. In such case, to confirm that non-amplification is due to the sequence of ITS region in ribosomal DNA but not due to a failed reaction, it is desirable to design primers not only for nonhomologous regions but also for homologous regions for internal control.

An amplification method other than PCR may be used as an identification method to confirm whether or not the ITS region in ribosomal DNA is amplified by the above specific primers. For instance, it is also possible to use the LAMP method (Eiken Chemical Co., Ltd.), the ICAN method (Takara Shuzo Co., Ltd.), or the like.

Classification by method other than nucleic acid amplification may be used. For example, a method comprising allowing single-strand nucleic acids to hybridize with each other and electrically detecting hybridization is a highly sensitive method. Thus, detection can be achieved without nucleic acid amplification. Sequence can be confirmed whether or not hybridization takes place by using a single-strand probe designed for a nonhomologous region as the above amplification method.

In addition, the RFLP (Restriction Fragment Length Polymorphism) method can be also used for identification as a method comprising comparing bases at 1 to several specific sites. Primers are designed by comparing some types of nucleotide sequences to be identified in order to find differences in terms of cleavage site of restriction enzymes among them so that amplified DNA fragments contain the cleavage sites. It is possible to determine sequence differences based on whether or not amplified DNA fragments are cleaved by the restriction enzyme. The cleavage or uncleavage can be readily determined by a usual electrophoresis method or the like.

Further, a method may comprise a combination of a plurality of different bases at the above specific sites to compare obtained patterns with each other. For instance, in the above primer method, a method, wherein a plurality of primer pairs is used, may be used. Alternatively, in the above RFLP method, relatively large regions are amplified, followed by cleavage with a plurality of restriction enzymes. Then, identification can be carried out based on cleavage patterns.

A test sample is not particularly limited as long as it is possible that the sample contains a plant of the genus Uncaria. For example, a test sample includes disrupted products, pulverized products, dried products and extracts of a plant of the genus Uncaria, herbal medicines and Chinese natural medicines containing thereof, and pharmaceutical products, foods, beverages and cosmetics containing such herbal medicines or Chinese natural medicines.

In addition, experiments, such as amplification of ITS region, PCR, cloning, nucleotide sequencing and nucleic acid chemical synthesis, used in the present invention can be generally performed by methods described in experimental protocols. As such an experimental protocol, Molecular Cloning, A laboratory manual (2001, 3rd Ed., Sambrook, J. & Russell, D W., Cold Spring Harbor Laboratory Press) is applied.

The present invention is hereafter described in greater detail with reference to the following examples, although the technical scope of the present invention is not limited thereto.

### EXAMPLES

### Example 1

### Materials

Mutation sites, which are useful for identification, were examined with the use of herbarium specimens which is identifiable based on the morphology. Table 3 lists examined samples. A plurality of samples of each species, excluding Uncaria homomella, were subjected to nucleotide sequencing of the ITS region and examined in terms of species-specific sites.

TABLE 3

| | DNA |
|---|---|
| Place | No. |
| U. rhynchophyllu (Miq.) Miq. | |
| Amatsu-kominato, Awa-gun, Chiba Pref., Japan | 0393 |
| Yawatano, Ito-city, Shizuoka Pref., Japan | 0392 |
| Isaibara, Nakamura-city, Kochi Pref., Japan | 0385 |
| Tongcheng, Hubei Prov., China | 0391 |
| Tongcheng, Hubei Prov., China | 0389 |
| Guanyingexian, Guanyang, Guangxi, China | 0395 |
| Huangjingxian, Gulin, Sichuan Prov., China | 0394 |
| Liangjiawan, Jinfushan, Nanchuan, Chongqing, | 0388 |
| Sichuan Prov., China | |
| Shimengou, Jinfushan, Nanchuan, Chongqing, | 0387 |
| Sichuan Prov., China | |
| Leishan, Guizhou Prov., China | 0264 |
| U. sinensis (Oliv.) Havil. | |
| Jiu long tunian, Emeishan, Sichuan Prov., China | 0700 |
| Miao tu ba, Nanchuan, Chongqing, Sichuan | 0701 |
| Prov., China | |
| Su bao, Leiguzhen, Beichuan, Sichuan Prov., | 0702 |
| China | |
| Beichuan, Sichuan Prov., China | 0712 |
| U. scandens (Smith) Hutchins. | |
| Mojiang Hani Autonomous County, Yunnan | 0263 |
| Prov., China | |
| Mojiang Hani Autonomous County, Yunnan | 0399 |
| Prov., China | |
| Yunnan Prov., China | 0732 |
| Dali, Yunnan Prov., China | 0704 |
| U. wangii How | |
| Simaoxian, Yunnan Prov., China | 0703 |
| Xishuangbanna, Yunnan Prov., China | 0708 |
| U. homomella Miq. | |
| Phongsaly, Laos | 0714 |
| U. hirsute Havil. | |
| Longzhouxian, Guangxi, China | 0397 |
| Ningmingxian, Guangxi, China | 0705 |
| Longzhouxian, Guangxi, China | 0731 |
| U. lancifolia Hutchins. | |
| Yadongxiang, Debaoxian, Guangxi, | 0402 |
| China | |
| Yunnan Prov., China | 0729 |
| Yunnan Prov., China | 0727 |
| U. sessilifructus Roxb. | |
| Simaoxian, Yunnan Prov., China | 0723 |
| Xishuangbanna, Yunnan Prov., | 0706 |
| China | |
| Xishuangbanna, Yunnan Prov., | 0725 |
| China | |
| Simaoxian, Yunnan Prov., China | 0698 |
| Simaoxian, Yunnan Prov., China | 0724 |
| Hekou, Yunnan Prov., China | 0699 |
| U. laevigata Wall. ex G. Don | |
| Xishuangbanna, Yunnan Prov., | 0386 |
| China | |
| Phongsaly, Laos | 0710 |
| U. macrophylla Wall. | |
| Simaoxian, Yunnan Prov., China | 0398 |
| Xishuangbanna, Yunnan Prov., | 0400 |
| China | |
| Xishuangbanna, Yunnan Prov., | 0401 |
| China = 38175? | |
| Guangxi, China | 0390 |
| Phongsaly, Laos | 0709 |
| U. yunnanensis K. C. Hsia | |
| Xishuangbanna, Yunnan Prov., | 0707 |
| China | |
| Luang Prabang, Laos | 0713 |

### Method

Approximately 200 mg of Leaves or pericarp was collected from each sample, followed by total DNA extraction with a DNeasy (registered trademark) Plant Mini Kit (Qiagen). The amplifiable lengths for PCR amplification of ITS regions were different depending on the states of the samples. Therefore, the regions were amplified by the steps as described below. In addition, with regard to samples that were difficult to amplify, purification of total DNA was attempted with the use of a Plasmid Mini Kit (Qiagen).

(1) ITS1, 5.8S, and ITS2 were collectively amplified with a primer set (ITS5 & ITS4) (White T. J. et al., 1990, Amplification and direct sequencing of fungal ribosomal RNA genes for phylogenetics, In PCR Protocols, A Guide to Methods and Applications, pp. 315-322, Academic Press, San Diego).

(SEQ ID NO: 54)

ITS5 (5′-GGA AGT AAA AGT CGT AAC AAG G-3′)

(SEQ ID NO: 55)

ITS4 (5′-TCC TCC GCT TAT TGA TAT GC-3′)

(2) ITS1 and ITS2 were separately amplified with a primer set (ITS5 & ITS2, ITS3 & ITS4) (White et al., 1990, described above).

(SEQ ID NO: 56)

ITS2 (5′-GCT GCG TTC TTC ATC GAT GC-3′)

(SEQ ID NO: 57)

ITS3 (5′-GCA TCG ATG AAG AAC GCA GC-3′)

(3) Primers were designed for regions conserved in a plant of the genus Uncaria based on the nucleotide sequence obtained by amplification and sequencing in steps (1) and (2). ITS1 and ITS2 were divided in half (4 fragments in total) and separately amplified. The primer set used is described below.

Amplification of ITS1: ITS5 & ITS-UncariaR2

(SEQ ID NO: 58)

(5′-GAG TTT TCC TTG GCG CGT TCC G-3′),
ITS-UncariaF1

(SEQ ID NO: 59)

(5′-CGC GCG GAA AAC GTA ACT CAA-3′) & ITS2
Amplification of ITS2: ITS3 & ITS-UncariaR1

(SEQ ID NO: 60)

(5′-ATT CAA CCA CCA CTC GTC GTG-3′),
ITS-UncariaF2

(SEQ ID NO: 61)

(5′-CTA AAT GAG AGT CCT CGG CG-3′) & ITS4.

The reaction solution used was as follows for each case: 5 μl of 10×GeneTaq-Buffer (Nippon Gene), 4 μl of dNTP mix (Nippon Gene), 1 μl of forward primer (ITS5: 10 pmol/μl), 1 μl of reverse primer (ITS4: 10 pmol/μl), 1.25 μl of template DNA, 0.25 μl Gene-Taq (Nippon Gene) ( ) 5 μl DMSO, and 32.5 μl D.D.W. (TaKaRa).

Reaction cycles are as follows: step (1): 94° C. for 1 minute, 48° C. for 2 minutes and 72° C. for 3 minutes×30 cycles, and 72° C. for 7 minutes×1 cycle; steps (2) and (3): 94° C. for 1 minute, 45° C. for 1 minute and 72° C. for 2 minutes×45 cycles, and 72° C. for 7 minutes×1 cycle.

Each PCR reaction product was degraded by electrophoresis with 2.0% agarose gel in accordance with DNA fragment size, followed by excision of DNA bands with target sizes from gel, and elution and purification with GFXTM PCR DNA and a Gel Band Purification Kit (Amersham biotech).

Purified products were sequenced with a BigDye Terminator Cycle Sequencing Kit ver.1.1 and a Model 3100 automated sequencer (Applied Bio Systems). Sequence was determined from both 5′- and 3′-terminals with primers used for amplification. The nucleotide sequence was finally concluded based on comparison thereof.

### Results

Among 4 primer sets used in step (3) for samples that were difficult to amplify, the highest amplification success rate was obtained for ITS-gUncariaF1 & ITS2 and the lowest amplification success rate was obtained for ITS5 & ITS-UncuriaR2.

The sizes of ITS 5.8 S and ITS2 for the examined plant of the genus Uncaria were 227-228 bp, 162-163 by and 219-220 bp, respectively. With regard to ITS1, genetic polymorphism within the genome was observed based on the deletion of a single base in two samples of Uncaria sessilifructus. Further, an irregular wave pattern was observed upon direct sequencing. With regard to 5.8S, both Uncaria macrophylla and Uncaria yunnanensis have deletion of a single base. With regard to the case of ITS2, Uncaria sessilifructus has insertion of a single base or genetic polymorphism within the genome regarding insertion at an identical site. In addition, Uncaria yunnanensis has insertion of a single base. Regarding Uncaria sessilifructus, even though amplification of the entire region is successfully carried out with a primer set of ITS5 & ITS4, it was necessary to carry out sequencing with a primer set of ITS2 & ITS3 for sequencing of the entire nucleotide sequence.

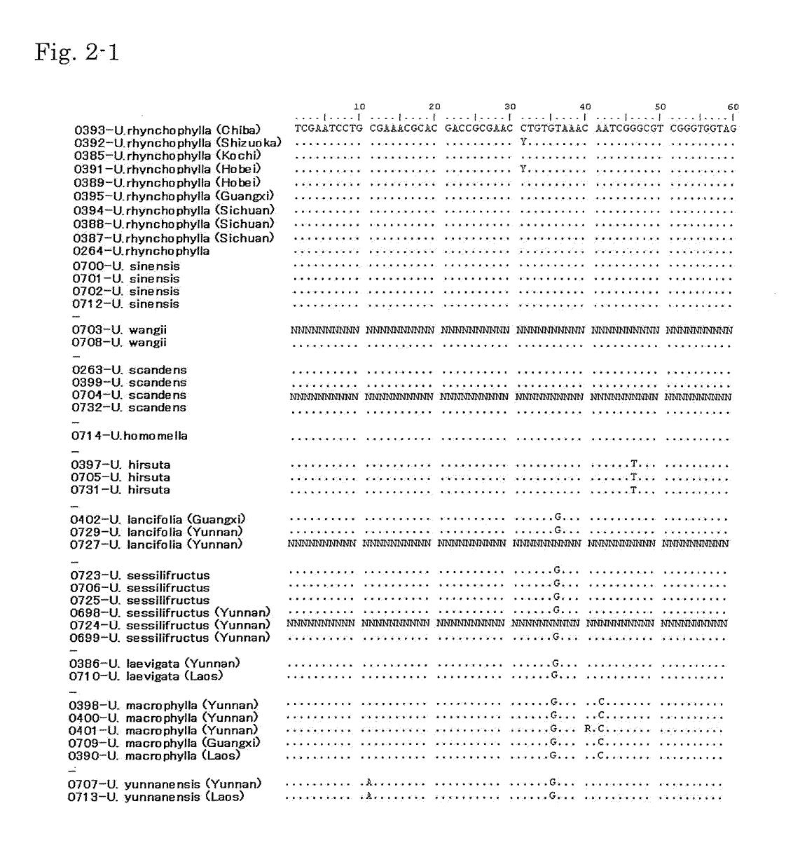

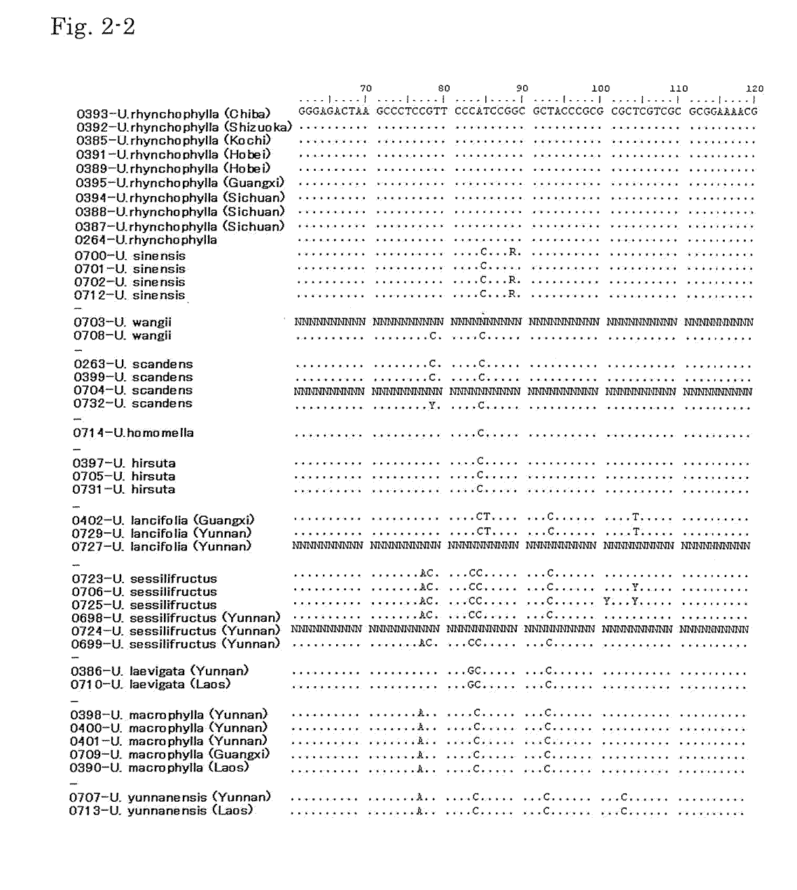

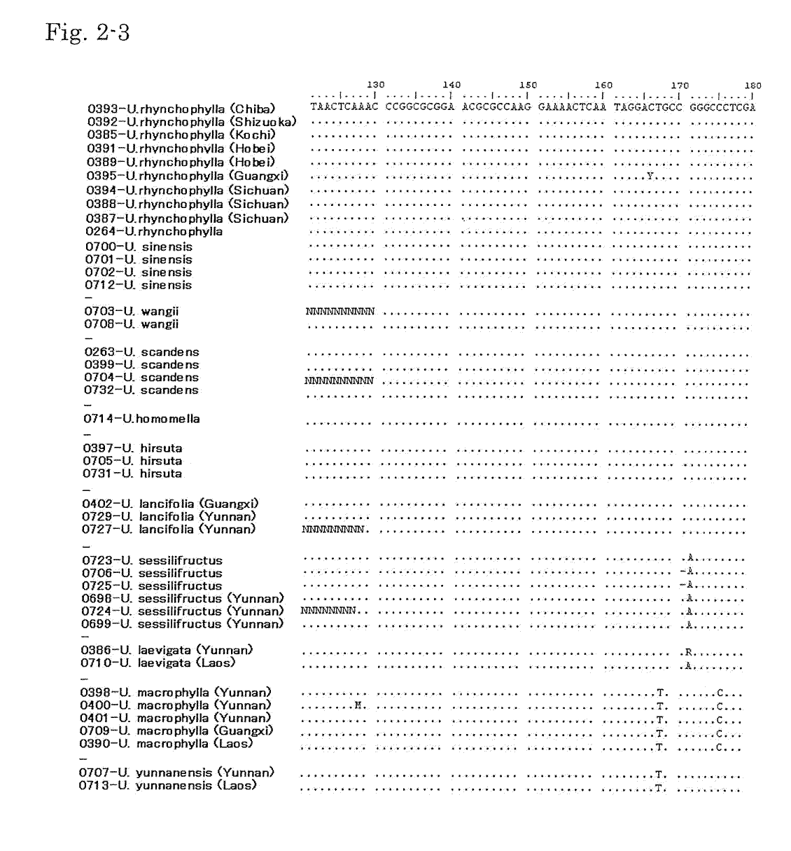

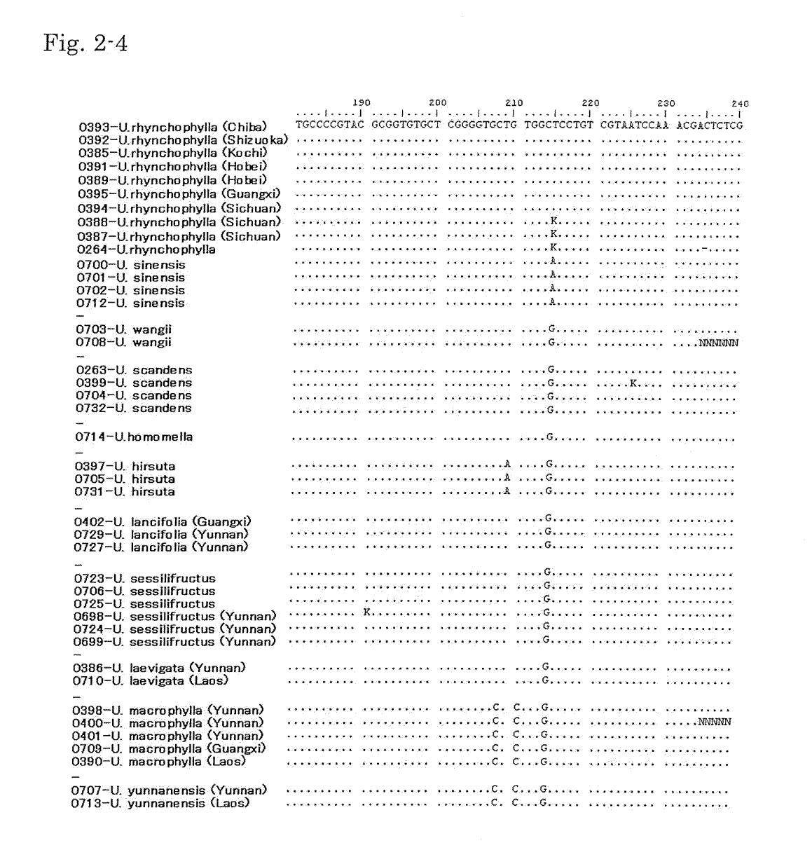

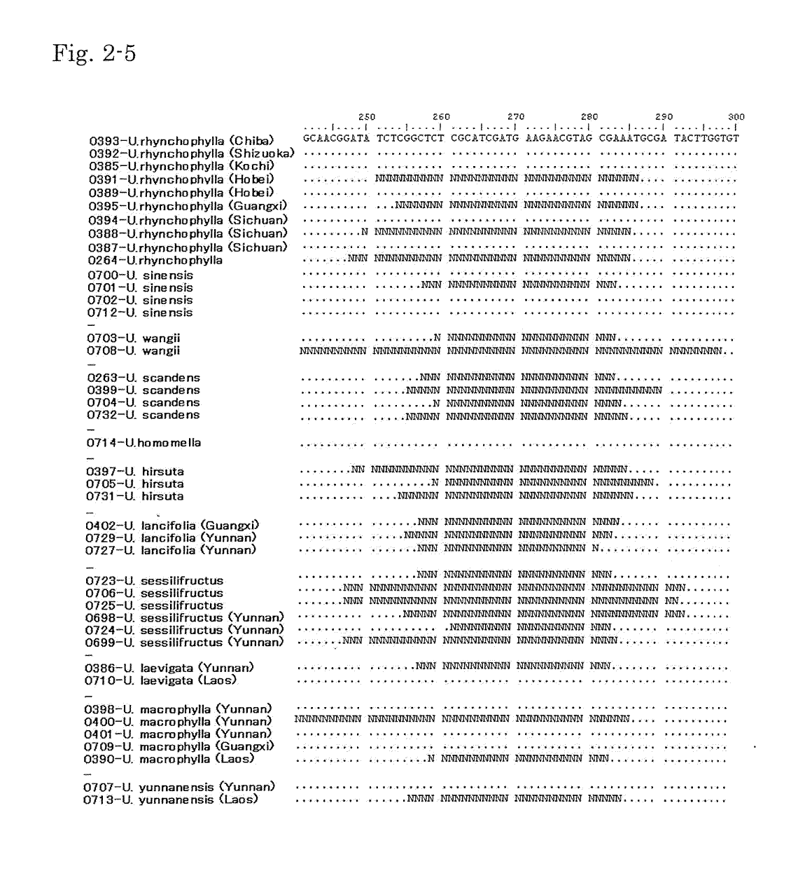

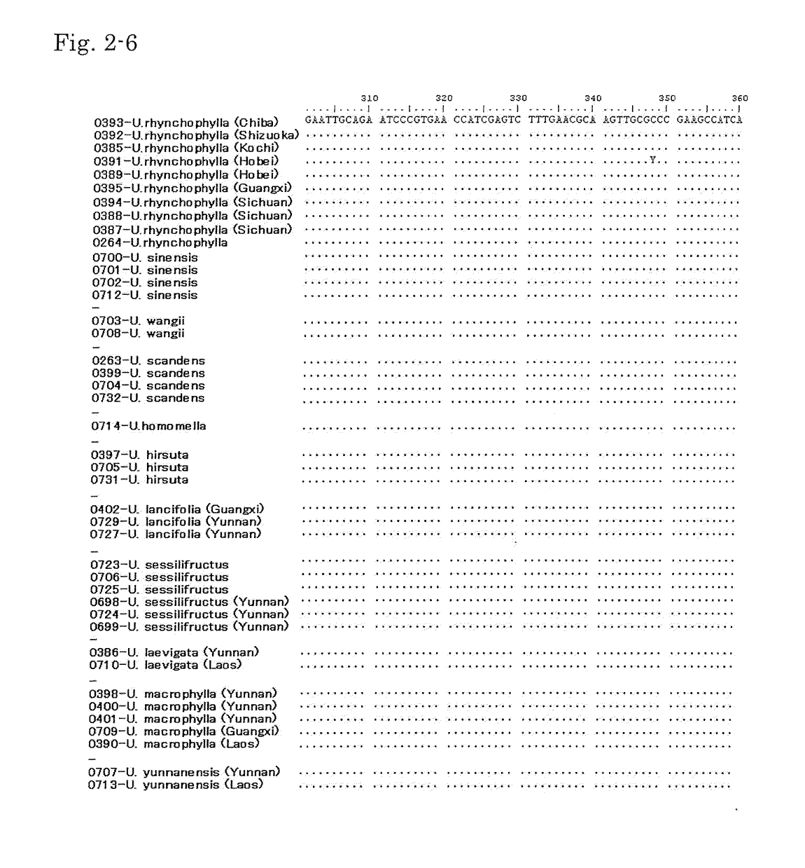

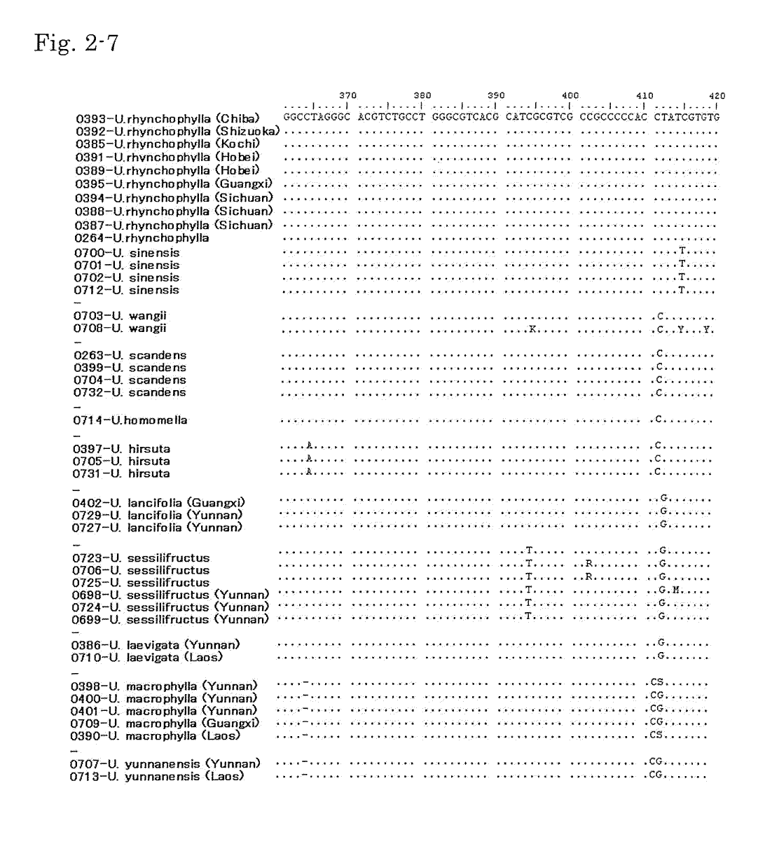

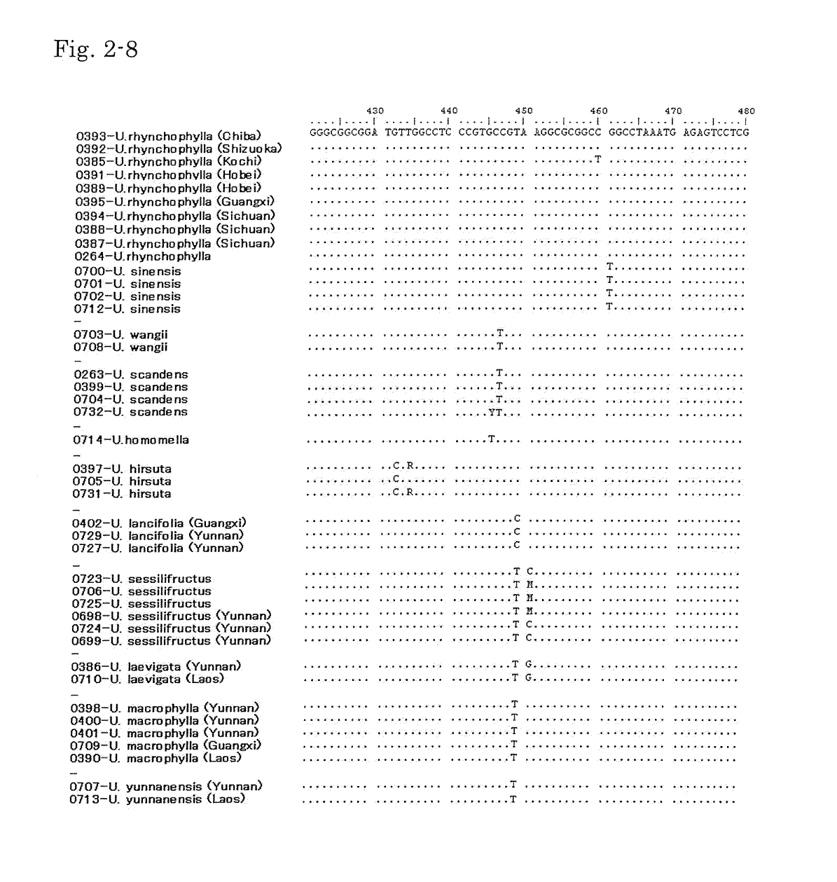

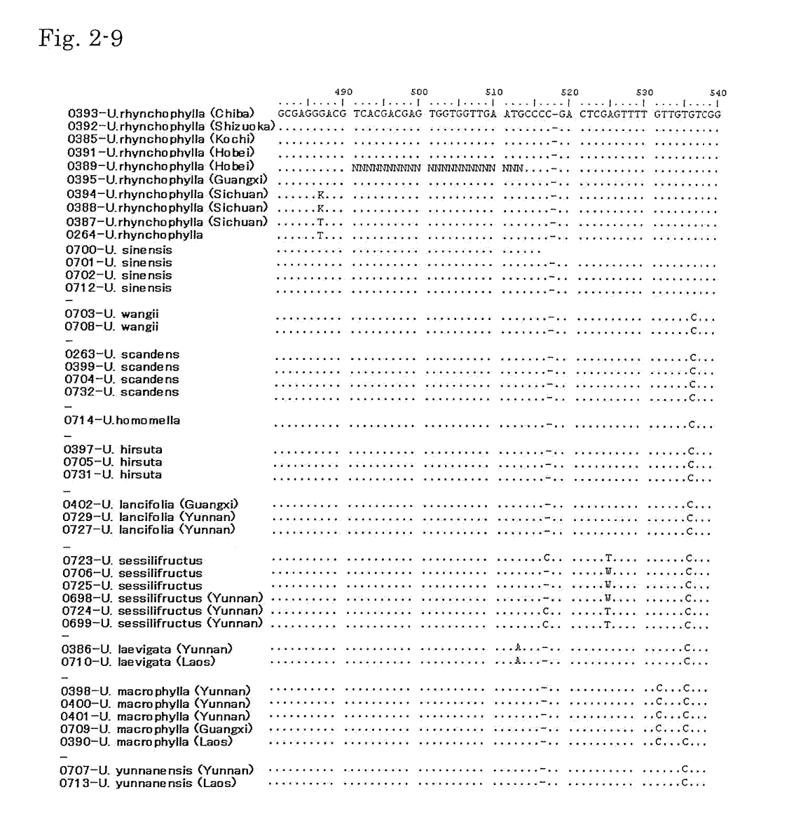

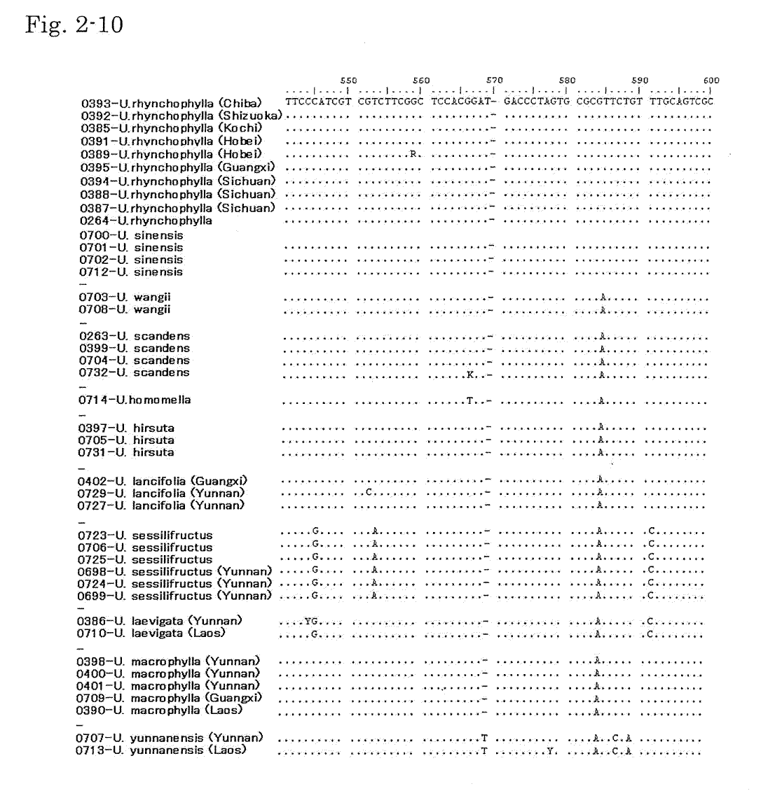

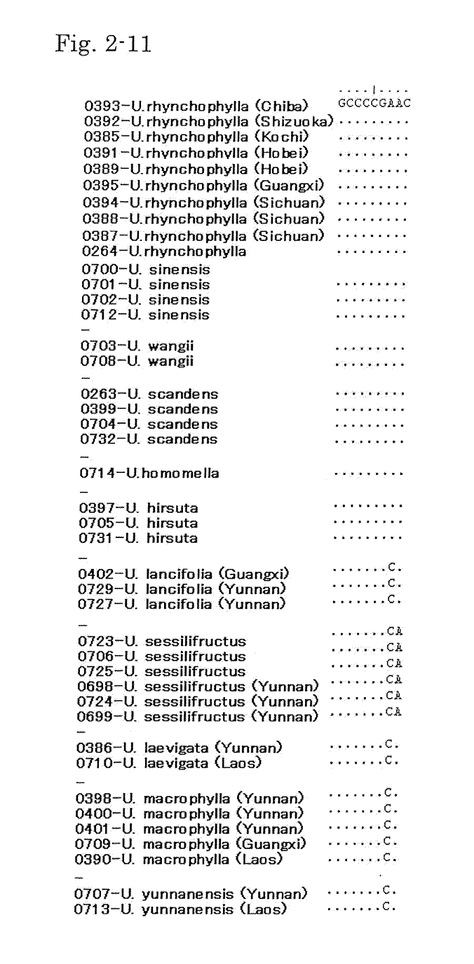

An examined region was found to have 64 mutations sites (FIG. 1). FIGS. 2-1 to 2-11 show results of nucleotide sequences alignment of ITS region obtained from samples of 42 species listed in Table 3 (corresponding to SEQ ID NOS: 12 to 53 from the top). Among the samples, complete base differences were observed in 45 out of 64 sites (positions 12, 37, 43, 47, 78, 79, 84, 85, 86, 94, 104, 105, 169, 172, 177, 209, 210, 211, 215, 365, 395, 412, 413, 415, 433, 446, 447, 450, 451, 461, 514, 518, 526, 532, 537, 546, 554, 567, 570, 585, 588, 590, 592, 608, and 609). In the remaining 19 sites, incomplete base substitution was exclusively observed. Such substitution was observed in only one or few samples for each site.

Each species was found to have specific and stable mutation patterns, excluding some exceptional cases. Therefore, it has been revealed that the ITS region is useful for identification between Diao-teng-gou original plants and closely-related species.

Based on the above results, it has been revealed that a Diao-teng-gou original plant and a plant belonging to the genus Uncaria that is closely related thereto can be identified based on their ITS region nucleotide sequences.

### Example 2

ITS region nucleotide sequences of Diao-teng-gou (herbal medicine) obtained in Huangjingxian, Gulin, Sichuan Prov. and Diao-teng-gou (herbal medicine) obtained from a company (Gong Si) in the production area in Sichuan Prov. were analyzed in order to identify the species of a plant of the genus Uncaria contained therein.

### Material

As the samples, two Diao-teng-gou samples obtained in Huangjingxian, Gulin, Sichuan Prov. and eight samples (two lots) of Diao-teng-gou obtained from Seito Ryusen Tennen Yakuhin Kaihatsu Yugen Koshi (Chengdu long quan tian ran yao pin kai fa you xian gong si) were used.

### Method

Total DNA was extracted from approximately 200 μg of a sample with a DNeasy (registered trademark) Plant Mini Kit (Qiagen). A primer set (ITS5 & ITS3) was used for PCR amplification of the ITS region. The composition of a reaction solution was as follows: 5 μl of 10× Buffer, 4 μl of 2.5 mM dNTP mix, 2.5 μl of 10 pmol/μl primer×2, 30.75 μl of 10% DMSO, 5 μl of template DNA and 0.25 μl of Gene-Taq. Reaction cycles were as follows: 94° C. for 4 minutes×1 cycle; 95° C. for 30 seconds, 70° C. for 15 seconds and 72° C. for 15 seconds×3 cycles; 95° C. for 30 seconds, 66° C. for 15 seconds and 72° C. for 15 seconds×3 cycles; 95° C. for 30 seconds, 62° C. for 15 seconds and 72° C. for 15 seconds×3 cycles; 95° C. for 30 seconds, 58° C. for 15 seconds and 72° C. for 15 seconds×3 cycles; 95° C. for 30 seconds, 54° C. for 15 seconds and 72° C. for 15 seconds×3 cycles; 95° C. for 30 seconds, 48° C. for 1 minute 30 seconds and 72° C. for 2 minutes 30 seconds×20 cycles; and 72° C. for 7 minutes×1 cycle.

Each PCR reaction product was subjected to electrophoresis with 2.0% agarose gel in accordance with DNA fragment size, followed by excision of DNA bands with target sizes and, elution and purification with GFXTM PCR DNA and a Gel Band Purification Kit (Amersham biotech).

Purified products were degraded by electrophoresis with a BigDye Terminator Cycle Sequencing Kit ver. 3.1 and a Model 3130xL Genetic Analyzer (Applied Biosystems). Sequence was determined from both 5′- and 3′-terminals with primers used for amplification. The nucleotide sequence was finally concluded based on comparison thereof.

### Results

As a result of analysis of the obtained nucleotide sequence of ITS region regarding each herbal medicine sample, the original plants of Diao-teng-gou obtained in Sichuan Pref. were identified as corresponding to Uncaria rhynchophylla and Uncaria sinensis. Thus, the distribution of Uncaria sinensis has been confirmed in Gulin in Sichuan Prov. In view of the above, it has been revealed that the plant species of the genus Uncaria can be identified based on the nucleotide sequence of the ITS region also with the use of herbal medicines as samples.

All publications, patents, and patent applications cited herein are incorporated herein by reference in their entirety.

1. A method of identifying the plant species of the genus Uncaria contained in a test sample, comprising comparing the nucleotide sequence of the ITS region in plant ribosomal DNA contained in a test sample with the nucleotide sequence of at least one ITS region selected from among the nucleotide sequences represented by SEQ ID NOS: 1 to 11.

2. The method according to claim 1, wherein the test sample is an herbal medicine.

3. The method according to claim 1, comprising identifying whether the plant of the genus Uncaria contained in a test sample is a species selected from the group consisting of Uncaria rhynchophylla, Uncaria sinensis, Uncaria macrophylla, Uncaria Uncaria scandens, Uncaria homomella, Uncaria hirsuta, Uncaria lancifolia, Uncaria sessilifructus, Uncaria laeyigata and Uncaria yunnanensis.

4. The method according to claim 1, comprising identifying whether or not the plant of the genus Uncaria contained in a test sample is a species selected from the group consisting of Uncaria rhynchophylla, Uncaria sinensis and Uncaria macrophylla.

5. The method according to claim 4, comprising comparing at least one base of the nucleotide sequence of the ITS region in plant ribosomal DNA contained in a test sample, selected from among bases located at positions corresponding to positions 12, 37, 43, 47, 78, 79, 84, 85, 86, 94, 104, 105, 169, 172, 177, 209, 210, 211, 215, 365, 395, 412, 413, 415, 433, 446, 447, 450, 451, 461, 514, 518, 526, 532, 537, 546, 554, 567, 570, 585, 588, 590, 592, 608 and 609 of the nucleotide sequence represented by SEQ ID NO: 1 used as a reference sequence with the selected nucleotide sequence of SEQ ID NO: 1.

6. The method according to claim 5, comprising comparing at least one base of the nucleotide sequence of the ITS region in plant ribosomal DNA contained in a test sample, selected from among bases located at positions corresponding to positions 12, 37, 43, 47, 79, 84, 85, 86, 94, 104, 105, 169, 172, 177, 209, 210, 211, 215, 365, 395, 412, 413, 415, 433, 446, 447, 450, 451, 461, 514, 518, 526, 532, 537, 546, 554, 567, 570, 585, 588, 590, 592, 608 and 609 of the nucleotide sequence represented by SEQ ID NO: 1 used as a reference sequence with the selected nucleotide sequence of SEQ ID NO: 1.

7. The method according to claim 6, comprising comparing at least one base of the nucleotide sequence of the ITS region in plant ribosomal DNA contained in a test sample, selected from among bases located at positions corresponding to positions 12, 37, 43, 85, 94, 104, 169, 177, 209, 211, 215, 412, 413, 415, 450, 461, 532, 537, 585, 588 and 608 of the nucleotide sequence represented by SEQ ID NO: 1 used as a reference sequence with the selected nucleotide sequence of SEQ ID NO: 1.


SEQUENCE LISTING (61 sequences)

SEQ ID NO: 1 - DNA, 607 bp, Uncaria rhynchophylla
tcgaatcctgcgaaacgcacgaccgcgaacytgtgtaaacaatcgggcgtcgggtggtaggggagactaagccctccgttcccatccggcgctacccgcgcgctcgtcgcgcggaaaacgtaactcaaacccggcgcggaacgcgccaaggaaaactcaataggaytgccgggccctcgatgccccgtacgcggtgtgctcggggtgctgtggckcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgyccgaagccatcaggcctagggcacgtctgcctgggcgtcacgcatcgcgtcgccgcccccacctatcgtgtggggcggcggatgttggcctcccgtgccgtaaggcgcggcyggcctaaatgagagtcctcggcgaggkacgtcacgacgagtggtggttgaatgccccgactcgagttttgttgtgtcggttcccatcgtcgtcttcgrctccacggatgaccctagtgcgcgttctgtttgcagtcgcgccccgaac

SEQ ID NO: 2 - DNA, 607 bp, Uncaria sinensis
tcgaatcctgcgaaacgcacgaccgcgaacctgtgtaaacaatcgggcgtcgggtggtaggggagactaagccctccgttcccacccgrcgctacccgcgcgctcgtcgcgcggaaaacgtaactcaaacccggcgcggaacgcgccaaggaaaactcaataggactgccgggccctcgatgccccgtacgcggtgtgctcggggtgctgtggcacctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctattgtgtggggcggcggatgttggcctcccgtgccgtaaggcgcggcctgcctaaatgagagtcctcggcgagggacgtcacgacgagtggtggttgaatgccccgactcgagttttgttgtgtcggttcccatcgtcgtcttcggctccacggatgaccctagtgcgcgttctgtttgcagtcgcgccccgaac

SEQ ID NO: 3 - DNA, 607 bp, Uncaria wangii
  misc_feature (235)..(298): n is a, c, g, or t
tcgaatcctgcgaaacgcacgaccgcgaacctgtgtaaacaatcgggcgtcgggtggtaggggagactaagccctccgctcccacccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggcgcctgtcgtaatccaaacgannnnnnnnnnnnnnnnnnnnnnnnnnnnnnnnnnnnnnnnnnnnnnnnnnnnnnnnnnnnnnnngtgaattgcagaatcccgtgaaccatcgagtctttgaacgcaagttgcgcccgaagccatcaggcctagggcacgtctgcctgggcgtcacgcatckcgtcgccgcccccacccatygtgyggggcggcggatgttggcctcccgtgctgtaaggcgcggccggcctaaatgagagtcctcggcgagggacgtcacgacgagtggtggttgaatgccccgactcgagttttgttgtgccggttcccatcgtcgtcttcggctccacggatgaccctagtgcgcgatctgtttgcagtcgcgccccgaac

SEQ ID NO: 4 - DNA, 607 bp, Uncaria scandens
  misc_feature (256)..(285): n is a, c, g, or t
tcgaatcctgcgaaacgcacgaccgcgaacctgtgtaaacaatcgggcgtcgggtggtaggggagactaagccctccgytcccacccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggcgcctgtcgtaatccaaacgackctcggcaacggatatctcgnnnnnnnnnnnnnnnnnnnnnnnnnnnnnntgcgatacttggtgtgaattgcagaatcccgtgaaccatcgagtctttgaacgcaagttgcgcccgaagccatcaggcctagggcacgtctgcctgggcgtcacgcatcgcgtcgccgcccccacccatcgtgtggggcggcggatgttggcctcccgtgytgtaaggcgcggccggcctaaatgagagtcctcggcgagggacgtcacgacgagtggtggttgaatgccccgactcgagttttgttgtgccggttcccatcgtcgtcttcggctccacgkatgaccctagtgcgcgatctgtttgcagtcgcgccccgaac

SEQ ID NO: 5 - DNA, 607 bp, Uncaria homomella
tcgaatcctgcgaaacgcacgaccgcgaacctgtgtaaacaatcgggcgtcgggtggtaggggagactaagccctccgttcccacccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggcgcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacccatcgtgtggggcggcggatgttggcctcccgtgtcgtaaggcgcggccggcctaaatgagagtcctcggcgagggacgtcacgacgagtggtggttgaatgccccgactcgagttttgttgtgccggttcccatcgtcgtcttcggctccacgtatgaccctagtgcgcgatctgtttgcagtcgcgccccgaac

SEQ ID NO: 6 - DNA, 607 bp, Uncaria hirsuta
  misc_feature (249)..(285): n is a, c, g, or t
tcgaatcctgcgaaacgcacgaccgcgaacctgtgtaaacaatcggtcgtcgggtggtaggggagactaagccctccgttcccacccggcgctacccgcgcgctcgtcgcgcggaaaacgtaactcaaacccggcgcggaacgcgccaaggaaaactcaataggactgccgggccctcgatgccccgtacgcggtgtgctcggggtgctatggcgcctgtcgtaatccaaacgactctcggcaacggannnnnnnnnnnnnnnnnnnnnnnnnnnnnnnnnnnnntgcgatacttggtgtgaattgcagaatcccgtgaaccatcgagtctttgaacgcaagttgcgcccgaagccatcaggccaagggcacgtctgcctgggcgtcacgcatcgcgtcgccgcccccacccatcgtgtggggcggcggatgctrgcctcccgtgccgtaaggcgcggccggcctaaatgagagtcctcggcgagggacgtcacgacgagtggtggttgaatgccccgactcgagttttgttgtgccggttcccatcgtcgtcttcggctccacggatgaccctagtgcgcgatctgtttgcagtcgcgccccgaac

SEQ ID NO: 7 - DNA, 607 bp, Uncaria lancifolia (Guangxi)
  misc_feature (258)..(284): n is a, c, g, or t
tcgaatcctgcgaaacgcacgaccgcgaacctgtgtgaacaatcgggcgtcgggtggtaggggagactaagccctccgttcccactcggcgctccccgcgcgcttgtcgcgcggaaaacgtaactcaaacccggcgcggaacgcgccaaggaaaactcaataggactgccgggccctcgatgccccgtacgcggtgtgctcggggtgctgtggcgcctgtcgtaatccaaacgactctcggcaacggatatctcggcnnnnnnnnnnnnnnnnnnnnnnnnnnnatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctgtcgtgtggggcggcggatgttggcctcccgtgccgtcaggcgcggccggcctaaatgagagtcctcggcgagggacgtcacgacgagtggtggttgaatgccccgactcgagttttgttgtgccggttcccatcgtcgycttcggctccacggatgaccctagtgcgcgatctgtttgcagtcgcgccccgacc

SEQ ID NO: 8 - DNA, 608 bp, Uncaria sessilifructus
  misc_feature (247)..(292): n is a, c, g, or t
tcgaatcctgcgaaacgcacgaccgcgaacctgtgtgaacaatcgggcgtcgggtggtaggggagactaagccctccactcccccccggcgctccccgcgygctygtcgcgcggaaaacgtaactcaaacccggcgcggaacgcgccaaggaaaactcaataggactgccgagccctcgatgccccgtackcggtgtgctcggggtgctgtggcgcctgtcgtaatccaaacgactctcggcaacggnnnnnnnnnnnnnnnnnnnnnnnnnnnnnnnnnnnnnnnnnnnnncttggtgtgaattgcagaatcccgtgaaccatcgagtctttgaacgcaagttgcgcccgaagccatcaggcctagggcacgtctgcctgggcgtcacgcatctcgtcgccrcccccacctgtcgtgtggggcggcggatgttggcctcccgtgccgttmggcgcggccggcctaaatgagagtcctcggcgagggacgtcacgacgagtggtggttgaatgcccccgactcgawttttgttgtgccggttcccgtcgtcgtattcggctccacggatgaccctagtgccggatctgttcgcagtcgcgccccgaca

SEQ ID NO: 9 - DNA, 607 bp, Uncaria laevigata (Yunnan)
  misc_feature (258)..(283): n is a, c, g, or t
tcgaatcctgcgaaacgcacgaccgcgaacctgtgtgaacaatcgggcgtcgggtggtaggggagactaagccctccgttcccgcccggcgctccccgcgcgctcgtcgcgcggaaaacgtaactcaaacccggcgcggaacgcgccaaggaaaactcaataggactgccgrgccctcgatgccccgtacgcggtgtgctcggggtgctgtggcgcctgtcgtaatccaaacgactctcggcaacggatatctcggcnnnnnnnnnnnnnnnnnnnnnnnnnnaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctgtcgtgtggggcggcggatgttggcctcccgtgccgttgggcgcggccggcctaaatgagagtcctcggcgagggacgtcacgacgagtggtggttgaatgacccgactcgagttttgttgtgccggttccygtcgtcgtcttcggctccacggatgaccctagtgcgcgatctgttcgcagtcgcgccccgacc

SEQ ID NO: 10 - DNA, 606 bp, Uncaria macrophylla (Yunnan)
tcgaatcctgcgaaacgcacgaccgcgaacctgtgtgaacraccgggcgtcgggtggtaggggagactaagccctccattcccacccggcgctccccgcgcgctcgtcgcgcggaaaacgtaactcaamcccggcgcggaacgcgccaaggaaaactcaataggactgtcgggcccccgatgccccgtacgcggtgtgctcggggtgccgcggcgcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggccagggcacgtctgcctgggcgtcacgcatcgcgtcgccgcccccacccstcgtgtggggcggcggatgttggcctcccgtgccgttaggcgcggccggcctaaatgagagtcctcggcgagggacgtcacgacgagtggtggttgaatgccccgactcgagttttgtcgtgccggttcccatcgtcgtcttcggctccacggatgaccctagtgcgcgatctgtttgcagtcgcgccccgacc

SEQ ID NO: 11 - DNA, 607 bp, Uncaria yunnanensis (Laos)
  misc_feature (257)..(285): n is a, c, g, or t
tcgaatcctgcaaaacgcacgaccgcgaacctgtgtgaacaatcgggcgtcgggtggtaggggagactaagccctccattcccacccggcgctccccgcgcgcccgtcgcgcggaaaacgtaactcaaacccggcgcggaacgcgccaaggaaaactcaataggactgtcgggccctcgatgccccgtacgcggtgtgctcggggtgccgcggcgcctgtcgtaatccaaacgactctcggcaacggatatctcggnnnnnnnnnnnnnnnnnnnnnnnnnnnnntgcgatacttggtgtgaattgcagaatcccgtgaaccatcgagtctttgaacgcaagttgcgcccgaagccatcaggccagggcacgtctgcctgggcgtcacgcatcgcgtcgccgcccccacccgtcgtgtggggcggcggatgttggcctcccgtgccgttaggcgcggccggcctaaatgagagtcctcggcgagggacgtcacgacgagtggtggttgaatgccccgactcgagttttgttgtgccggttcccatcgtcgtcttcggctccacggattgaccctagygcgcgatccgattgcagtcgcgccccgacc

SEQ ID NO: 12 - DNA, 607 bp, Uncaria rhynchophylla (Chiba)
tcgaatcctgcgaaacgcacgaccgcgaacctgtgtaaacaatcgggcgtcgggtggtaggggagactaagccctccgttcccatccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggctcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctatcgtgtggggcggcggatgttggcctcccgtgccgtaaggcgcggccggcctaaatgagagtcctcggcgagggacgtcacgacgagtggtggttgaatgccccgactcgagttttgttgtgtcggttcccatcgtcgtcttcggctccacggatgaccctagtgcgcgttctgtttgcagtcgcgccccgaac

SEQ ID NO: 13 - DNA, 607 bp, Uncaria rhynchophylla (Shizuoka)
tcgaatcctgcgaaacgcacgaccgcgaacytgtgtaaacaatcgggcgtcgggtggtaggggagactaagccctccgttcccatccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggctcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctatcgtgtggggcggcggatgttggcctcccgtgccgtaaggcgcggccggcctaaatgagagtcctcggcgagggacgtcacgacgagtggtggttgaatgccccgactcgagttttgttgtgtcggttcccatcgtcgtcttcggctccacggatgaccctagtgcgcgttctgtttgcagtcgcgccccgaac

SEQ ID NO: 14 - DNA, 607 bp, Uncaria rhynchophylla (Kochi)
tcgaatcctgcgaaacgcacgaccgcgaacctgtgtaaacaatcgggcgtcgggtggtaggggagactaagccctccgttcccatccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggctcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctatcgtgtggggcggcggatgttggcctcccgtgccgtaaggcgcggctggcctaaatgagagtcctcggcgagggacgtcacgacgagtggtggttgaatgccccgactcgagttttgttgtgtcggttcccatcgtcgtcttcggctccacggatgaccctagtgcgcgttctgtttgcagtcgcgccccgaac

SEQ ID NO: 15 - DNA, 607 bp, Uncaria rhynchophylla (Hobei)
  misc_feature (251)..(286): n is a, c, g, or t
tcgaatcctgcgaaacgcacgaccgcgaacytgtgtaaacaatcgggcgtcgggtggtaggggagactaagccctccgttcccatccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggctcctgtcgtaatccaaacgactctcggcaacggatannnnnnnnnnnnnnnnnnnnnnnnnnnnnnnnnnnngcgatacttggtgtgaattgcagaatcccgtgaaccatcgagtctttgaacgcaagttgcgyccgaagccatcaggcctagggcacgtctgcctgggcgtcacgcatcgcgtcgccgcccccacctatcgtgtggggcggcggatgttggcctcccgtgccgtaaggcgcggccggcctaaatgagagtcctcggcgagggacgtcacgacgagtggtggttgaatgccccgactcgagttttgttgtgtcggttcccatcgtcgtcttcggctccacggatgaccctagtgcgcgttctgtttgcagtcgcgccccgaac

SEQ ID NO: 16 - DNA, 607 bp, Uncaria rhynchophylla (Hobei)
  misc_feature (491)..(513): n is a, c, g, or t
tcgaatcctgcgaaacgcacgaccgcgaacctgtgtaaacaatcgggcgtcgggtggtaggggagactaagccctccgttcccatccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggctcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctatcgtgtggggcggcggatgttggcctcccgtgccgtaaggcgcggccggcctaaatgagagtcctcggcgagggacgnnnnnnnnnnnnnnnnnnnnnnnccccgactcgagttttgttgtgtcggttcccatcgtcgtcttcgrctccacggatgaccctagtgcgcgttctgtttgcagtcgcgccccgaac

SEQ ID NO: 17 - DNA, 607 bp, Uncaria rhynchophylla (Guangxi)
  misc_feature (254)..(286): n is a, c, g, or t
tcgaatcctgcgaaacgcacgaccgcgaacctgtgtaaacaatcgggcgtcgggtggtaggggagactaagccctccgttcccatccggcgctacccgcgcgctcgtcgcgcggaaaacgtaactcaaacccggcgcggaacgcgccaaggaaaactcaataggaytgccgggccctcgatgccccgtacgcggtgtgctcggggtgctgtggctcctgtcgtaatccaaacgactctcggcaacggatatctnnnnnnnnnnnnnnnnnnnnnnnnnnnnnnnnngcgatacttggtgtgaattgcagaatcccgtgaaccatcgagtctttgaacgcaagttgcgcccgaagccatcaggcctagggcacgtctgcctgggcgtcacgcatcgcgtcgccgcccccacctatcgtgtggggcggcggatgttggcctcccgtgccgtaaggcgcggccggcctaaatgagagtcctcggcgagggacgtcacgacgagtggtggttgaatgccccgactcgagttttgttgtgtcggttcccatcgtcgtcttcggctccacggatgaccctagtgcgcgttctgtttgcagtcgcgccccgaac

SEQ ID NO: 18 - DNA, 607 bp, Uncaria rhynchophylla (Sichuan)
tcgaatcctgcgaaacgcacgaccgcgaacctgtgtaaacaatcgggcgtcgggtggtaggggagactaagccctccgttcccatccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggctcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctatcgtgtggggcggcggatgttggcctcccgtgccgtaaggcgcggccggcctaaatgagagtcctcggcgaggkacgtcacgacgagtggtggttgaatgccccgactcgagttttgttgtgtcggttcccatcgtcgtcttcggctccacggatgaccctagtgcgcgttctgtttgcagtcgcgccccgaac

SEQ ID NO: 19 - DNA, 607 bp, Uncaria rhynchophylla (Sichuan)
  misc_feature (250)..(285): n is a, c, g, or t
tcgaatcctgcgaaacgcacgaccgcgaacctgtgtaaacaatcgggcgtcgggtggtaggggagactaagccctccgttcccatccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggckcctgtcgtaatccaaacgactctcggcaacggatnnnnnnnnnnnnnnnnnnnnnnnnnnnnnnnnnnnntgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctatcgtgtggggcggcggatgttggcctcccgtgccgtaaggcgcggccggcctaaatgagagtcctcggcgaggkacgtcacgacgagtggtggttgaatgccccgactcgagttttgttgtgtcggttcccatcgtcgtcttcggctccacggatgaccctagtgcgcgttctgtttgcagtcgcgccccgaac

SEQ ID NO: 20 - DNA, 607 bp, Uncaria rhynchophylla (Sichuan)
tcgaatcctgcgaaacgcacgaccgcgaacctgtgtaaacaatcgggcgtcgggtggtaggggagactaagccctccgttcccatccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggckcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctatcgtgtggggcggcggatgttggcctcccgtgccgtaaggcgcggccggcctaaatgagagtcctcggcgaggtacgtcacgacgagtggtggttgaatgccccgactcgagttttgttgtgtcggttcccatcgtcgtcttcggctccacggatgaccctagtgcgcgttctgtttgcagtcgcgccccgaac

SEQ ID NO: 21 - DNA, 606 bp, Uncaria rhynchophylla
  misc_feature (247)..(284): n is a, c, g, or t
tcgaatcctgcgaaacgcacgaccgcgaacctgtgtaaacaatcgggcgtcgggtggtaggggagactaagccctccgttcccatccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggckcctgtcgtaatccaaacgatctcggcaacggnnnnnnnnnnnnnnnnnnnnnnnnnnnnnnnnnnnnnntgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctatcgtgtggggcggcggatgttggcctcccgtgccgtaaggcgcggccggcctaaatgagagtcctcggcgaggtacgtcacgacgagtggtggttgaatgccccgactcgagttttgttgtgtcggttcccatcgtcgtcttcggctccacggatgaccctagtgcgcgttctgtttgcagtcgcgccccgaac

SEQ ID NO: 22 - DNA, 516 bp, Uncaria sinensis
tcgaatcctgcgaaacgcacgaccgcgaacctgtgtaaacaatcgggcgtcgggtggtaggggagactaagccctccgttcccacccgrcgctacccgcgcgctcgtcgcgcggaaaacgtaactcaaacccggcgcggaacgcgccaaggaaaactcaataggactgccgggccctcgatgccccgtacgcggtgtgctcggggtgctgtggcacctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctattgtgtggggcggcggatgttggcctcccgtgccgtaaggcgcggcctgcctaaatgagagtcctcggcgagggacgtcacgacgagtggtggttgaatgccc

SEQ ID NO: 23 - DNA, 607 bp, Uncaria sinensis
  misc_feature (258)..(283): n is a, c, g, or t
tcgaatcctgcgaaacgcacgaccgcgaacctgtgtaaacaatcgggcgtcgggtggtaggggagactaagccctccgttcccacccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggcacctgtcgtaatccaaacgactctcggcaacggatatctcggcnnnnnnnnnnnnnnnnnnnnnnnnnnaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctattgtgtggggcggcggatgttggcctcccgtgccgtaaggcgcggcctgcctaaatgagagtcctcggcgagggacgtcacgacgagtggtggttgaatgccccgactcgagttttgttgtgtcggttcccatcgtcgtcttcggctccacggatgaccctagtgcgcgttctgtttgcagtcgcgccccgaac

SEQ ID NO: 24 - DNA, 607 bp, Uncaria sinensis; identical to SEQ ID NO: 2

SEQ ID NO: 25 - DNA, 607 bp, Uncaria sinensis; identical to SEQ ID NO: 2

SEQ ID NO: 26 - DNA, 607 bp, Uncaria wangii
  misc_feature (1)..(130): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccggcgcggaacgcgccaaggaaaactcaataggactgccgggccctcgatgccccgtacgcggtgtgctcggggtgctgtggcgcctgtcgtaatccaaacgactctcggcaacggatatctcggctcnnnnnnnnnnnnnnnnnnnnnnnnaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacccatcgtgtggggcggcggatgttggcctcccgtgctgtaaggcgcggccggcctaaatgagagtcctcggcgagggacgtcacgacgagtggtggttgaatgccccgactcgagttttgttgtgccggttcccatcgtcgtcttcggctccacggatgaccctagtgcgcgatctgtttgcagtcgcgccccgaac

SEQ ID NO: 27 - DNA, 607 bp, Uncaria wangii; identical to SEQ ID NO: 3
  misc_feature (235)..(298): n is a, c, g, or t

SEQ ID NO: 28 - DNA, 607 bp, Uncaria scandens
  misc_feature (258)..(283): n is a, c, g, or t
tcgaatcctgcgaaacgcacgaccgcgaacctgtgtaaacaatcgggcgtcgggtggtaggggagactaagccctccgctcccacccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggcgcctgtcgtaatccaaacgactctcggcaacggatatctcggcnnnnnnnnnnnnnnnnnnnnnnnnnnaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacccatcgtgtggggcggcggatgttggcctcccgtgctgtaaggcgcggccggcctaaatgagagtcctcggcgagggacgtcacgacgagtggtggttgaatgccccgactcgagttttgttgtgccggttcccatcgtcgtcttcggctccacggatgaccctagtgcgcgatctgtttgcagtcgcgccccgaac

SEQ ID NO: 29 - DNA, 607 bp, Uncaria scandens
  misc_feature (256)..(290): n is a, c, g, or t
tcgaatcctgcgaaacgcacgaccgcgaacctgtgtaaacaatcgggcgtcgggtggtaggggagactaagccctccgctcccacccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggcgcctgtcgtaakccaaacgactctcggcaacggatatctcgnnnnnnnnnnnnnnnnnnnnnnnnnnnnnnnnnnntacttggtgtgaattgcagaatcccgtgaaccatcgagtctttgaacgcaagttgcgcccgaagccatcaggcctagggcacgtctgcctgggcgtcacgcatcgcgtcgccgcccccacccatcgtgtggggcggcggatgttggcctcccgtgctgtaaggcgcggccggcctaaatgagagtcctcggcgagggacgtcacgacgagtggtggttgaatgccccgactcgagttttgttgtgccggttcccatcgtcgtcttcggctccacggatgaccctagtgcgcgatctgtttgcagtcgcgccccgaac

SEQ ID NO: 30 - DNA, 607 bp, Uncaria scandens
  misc_feature (1)..(130): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccggcgcggaacgcgccaaggaaaactcaataggactgccgggccctcgatgccccgtacgcggtgtgctcggggtgctgtggcgcctgtcgtaatccaaacgactctcggcaacggatatctcggctcnnnnnnnnnnnnnnnnnnnnnnnnnatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacccatcgtgtggggcggcggatgttggcctcccgtgctgtaaggcgcggccggcctaaatgagagtcctcggcgagggacgtcacgacgagtggtggttgaatgccccgactcgagttttgttgtgccggttcccatcgtcgtcttcggctccacggatgaccctagtgcgcgatctgtttgcagtcgcgccccgaac

SEQ ID NO: 31 - DNA, 607 bp, Uncaria scandens
  misc_feature (256)..(285): n is a, c, g, or t
tcgaatcctgcgaaacgcacgaccgcgaacctgtgtaaacaatcgggcgtcgggtggtaggggagactaagccctccgytcccacccggcgctacccgcgcgctcgtcgcgcggaaaacgtaactcaaacccggcgcggaacgcgccaaggaaaactcaataggactgccgggccctcgatgccccgtacgcggtgtgctcggggtgctgtggcgcctgtcgtaatccaaacgactctcggcaacggatatctcgnnnnnnnnnnnnnnnnnnnnnnnnnnnnnntgcgatacttggtgtgaattgcagaatcccgtgaaccatcgagtctttgaacgcaagttgcgcccgaagccatcaggcctagggcacgtctgcctgggcgtcacgcatcgcgtcgccgcccccacccatcgtgtggggcggcggatgttggcctcccgtgytgtaaggcgcggccggcctaaatgagagtcctcggcgagggacgtcacgacgagtggtggttgaatgccccgactcgagttttgttgtgccggttcccatcgtcgtcttcggctccacgkatgaccctagtgcgcgatctgtttgcagtcgcgccccgaac

SEQ ID NO: 32 - DNA, 607 bp, Uncaria homomella; identical to SEQ ID NO: 5

SEQ ID NO: 33 - DNA, 607 bp, Uncaria hirsuta; identical to SEQ ID NO: 6
  misc_feature (249)..(285): n is a, c, g, or t

SEQ ID NO: 34 - DNA, 607 bp, Uncaria hirsuta
  misc_feature (260)..(289): n is a, c, g, or t
tcgaatcctgcgaaacgcacgaccgcgaacctgtgtaaacaatcggtcgtcgggtggtaggggagactaagccctccgttcccacccggcgctacccgcgcgctcgtcgcgcggaaaacgtaactcaaacccggcgcggaacgcgccaaggaaaactcaataggactgccgggccctcgatgccccgtacgcggtgtgctcggggtgctatggcgcctgtcgtaatccaaacgactctcggcaacggatatctcggctcnnnnnnnnnnnnnnnnnnnnnnnnnnnnnnatacttggtgtgaattgcagaatcccgtgaaccatcgagtctttgaacgcaagttgcgcccgaagccatcaggccaagggcacgtctgcctgggcgtcacgcatcgcgtcgccgcccccacccatcgtgtggggcggcggatgctggcctcccgtgccgtaaggcgcggccggcctaaatgagagtcctcggcgagggacgtcacgacgagtggtggttgaatgccccgactcgagttttgttgtgccggttcccatcgtcgtcttcggctccacggatgaccctagtgcgcgatctgtttgcagtcgcgccccgaac

SEQ ID NO: 35 - DNA, 607 bp, Uncaria hirsuta
  misc_feature (255)..(286): n is a, c, g, or t
tcgaatcctgcgaaacgcacgaccgcgaacctgtgtaaacaatcggtcgtcgggtggtaggggagactaagccctccgttcccacccggcgctacccgcgcgctcgtcgcgcggaaaacgtaactcaaacccggcgcggaacgcgccaaggaaaactcaataggactgccgggccctcgatgccccgtacgcggtgtgctcggggtgctatggcgcctgtcgtaatccaaacgactctcggcaacggatatctcnnnnnnnnnnnnnnnnnnnnnnnnnnnnnnnngcgatacttggtgtgaattgcagaatcccgtgaaccatcgagtctttgaacgcaagttgcgcccgaagccatcaggccaagggcacgtctgcctgggcgtcacgcatcgcgtcgccgcccccacccatcgtgtggggcggcggatgctrgcctcccgtgccgtaaggcgcggccggcctaaatgagagtcctcggcgagggacgtcacgacgagtggtggttgaatgccccgactcgagttttgttgtgccggttcccatcgtcgtcttcggctccacggatgaccctagtgcgcgatctgtttgcagtcgcgccccgaac

SEQ ID NO: 36 - DNA, 607 bp, Uncaria lancifolia (Guangxi)
  misc_feature (258)..(284): n is a, c, g, or t
tcgaatcctgcgaaacgcacgaccgcgaacctgtgtgaacaatcgggcgtcgggtggtaggggagactaagccctccgttcccactcggcgctccccgcgcgcttgtcgcgcggaaaacgtaactcaaacccggcgcggaacgcgccaaggaaaactcaataggactgccgggccctcgatgccccgtacgcggtgtgctcggggtgctgtggcgcctgtcgtaatccaaacgactctcggcaacggatatctcggcnnnnnnnnnnnnnnnnnnnnnnnnnnnatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctgtcgtgtggggcggcggatgttggcctcccgtgccgtcaggcgcggccggcctaaatgagagtcctcggcgagggacgtcacgacgagtggtggttgaatgccccgactcgagttttgttgtgccggttcccatcgtcgtcttcggctccacggatgaccctagtgcgcgatctgtttgcagtcgcgccccgacc

SEQ ID NO: 37 - DNA, 607 bp, Uncaria lancifolia (Yunnan)
  misc_feature (256)..(283): n is a, c, g, or t
tcgaatcctgcgaaacgcacgaccgcgaacctgtgtgaacaatcgggcgtcgggtggtaggggagactaagccctccgttcccactcggcgctccccgcgcgcttgtcgcgcggaaaacgtaactcaaacccggcgcggaacgcgccaaggaaaactcaataggactgccgggccctcgatgccccgtacgcggtgtgctcggggtgctgtggcgcctgtcgtaatccaaacgactctcggcaacggatatctcgnnnnnnnnnnnnnnnnnnnnnnnnnnnnaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctgtcgtgtggggcggcggatgttggcctcccgtgccgtcaggcgcggccggcctaaatgagagtcctcggcgagggacgtcacgacgagtggtggttgaatgccccgactcgagttttgttgtgccggttcccatcgtcgccttcggctccacggatgaccctagtgcgcgatctgtttgcagtcgcgccccgacc

SEQ ID NO: 38 - DNA, 607 bp, Uncaria lancifolia (Yunnan)
  misc_feature (1)..(129): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccggcgcggaacgcgccaaggaaaactcaataggactgccgggccctcgatgccccgtacgcggtgtgctcggggtgctgtggcgcctgtcgtaatccaaacgactctcggcaacggatatctcggcnnnnnnnnnnnnnnnnnnnnnnnngaaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctgtcgtgtggggcggcggatgttggcctcccgtgccgtcaggcgcggccggcctaaatgagagtcctcggcgagggacgtcacgacgagtggtggttgaatgccccgactcgagttttgttgtgccggttcccatcgtcgtcttcggctccacggatgaccctagtgcgcgatctgtttgcagtcgcgccccgacc

SEQ ID NO: 39 - DNA, 608 bp, Uncaria sessilifructus
  misc_feature (258)..(283): n is a, c, g, or t
tcgaatcctgcgaaacgcacgaccgcgaacctgtgtgaacaatcgggcgtcgggtggtaggggagactaagccctccactcccccccggcgctccccgcgcgctcgtcgcgcggaaaacgtaactcaaacccggcgcggaacgcgccaaggaaaactcaataggactgccgagccctcgatgccccgtacgcggtgtgctcggggtgctgtggcgcctgtcgtaatccaaacgactctcggcaacggatatctcggcnnnnnnnnnnnnnnnnnnnnnnnnnnaatgcgatacttggtgtgaattgcagaatcccgtgaaccatcgagtctttgaacgcaagttgcgcccgaagccatcaggcctagggcacgtctgcctgggcgtcacgcatctcgtcgccgcccccacctgtcgtgtggggcggcggatgttggcctcccgtgccgttcggcgcggccggcctaaatgagagtcctcggcgagggacgtcacgacgagtggtggttgaatgcccccgactcgatttttgttgtgccggttcccgtcgtcgtattcggctccacggatgaccctagtgcgcgatctgttcgcagtcgcgccccgaca

SEQ ID NO: 40 - DNA, 606 bp, Uncaria sessilifructus
  misc_feature (247)..(292): n is a, c, g, or t
tcgaatcctgcgaaacgcacgaccgcgaacctgtgtgaacaatcgggcgtcgggtggtaggggagactaagccctccactcccccccggcgctccccgcgcgctygtcgcgcggaaaacgtaactcaaacccggcgcggaacgcgccaaggaaaactcaataggactgccagccctcgatgccccgtacgcggtgtgctcggggtgctgtggcgcctgtcgtaatccaaacgactctcggcaacggnnnnnnnnnnnnnnnnnnnnnnnnnnnnnnnnnnnnnnnnnnnnnnttggtgtgaattgcagaatcccgtgaaccatcgagtctttgaacgcaagttgcgcccgaagccatcaggcctagggcacgtctgcctgggcgtcacgcatctcgtcgccrcccccacctgtcgtgtggggcggcggatgttggcctcccgtgccgttmggcgcggccggcctaaatgagagtcctcggcgagggacgtcacgacgagtggtggttgaatgccccgactcgawttttgttgtgccggttcccgtcgtcgtattcggctccacggatgaccctagtgcgcgatctgttcgcagtcgcgccccgaca

SEQ ID NO: 41 - DNA, 606 bp, Uncaria sessilifructus
  misc_feature (247)..(291): n is a, c, g, or t
tcgaatcctgcgaaacgcacgaccgcgaacctgtgtgaacaatcgggcgtcgggtggtaggggagactaagccctccactcccccccggcgctccccgcgygctygtcgcgcggaaaacgtaactcaaacccggcgcggaacgcgccaaggaaaactcaataggactgccagccctcgatgccccgtacgcggtgtgctcggggtgctgtggcgcctgtcgtaatccaaacgactctcggcaacggnnnnnnnnnnnnnnnnnnnnnnnnnnnnnnnnnnnnnnnnnnnnncttggtgtgaattgcagaatcccgtgaaccatcgagtctttgaacgcaagttgcgcccgaagccatcaggcctagggcacgtctgcctgggcgtcacgcatctcgtcgccrcccccacctgtcgtgtggggcggcggatgttggcctcccgtgccgttmggcgcggccggcctaaatgagagtcctcggcgagggacgtcacgacgagtggtggttgaatgccccgactcgawttttgttgtgccggttcccgtcgtcgtattcggctccacggatgaccctagtgcgcgatctgttcgcagtcgcgccccgaca

SEQ ID NO: 42 - DNA, 607 bp, Uncaria sessilifructus (Yunnan)
  misc_feature (256)..(293): n is a, c, g, or t
tcgaatcctgcgaaacgcacgaccgcgaacctgtgtgaacaatcgggcgtcgggtggtaggggagactaagccctccactcccccccggcgctccccgcgcgctcgtcgcgcggaaaacgtaactcaaacccggcgcggaacgcgccaaggaaaactcaataggactgccgagccctcgatgccccgtackcggtgtgctcggggtgctgtggcgcctgtcgtaatccaaacgactctcggcaacggatatctcgnnnnnnnnnnnnnnnnnnnnnnnnnnnnnnnnnnnnnnttggtgtgaattgcagaatcccgtgaaccatcgagtctttgaacgcaagttgcgcccgaagccatcaggcctagggcacgtctgcctgggcgtcacgcatctcgtcgccgcccccacctgtmgtgtggggcggcggatgttggcctcccgtgccgttmggcgcggccggcctaaatgagagtcctcggcgagggacgtcacgacgagtggtggttgaatgccccgactcgawttttgttgtgccggttcccgtcgtcgtattcggctccacggatgaccctagtgcgcgatctgttcgcagtcgcgccccgaca

SEQ ID NO: 43 - DNA, 608 bp, Uncaria sessilifructus (Yunnan)
  misc_feature (1)..(128): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacccggcgcggaacgcgccaaggaaaactcaataggactgccgagccctcgatgccccgtacgcggtgtgctcggggtgctgtggcgcctgtcgtaatccaaacgactctcggcaacggatatctcggctctcnnnnnnnnnnnnnnnnnnnnnnaatgcgatacttggtgtgaattgcagaatcccgtgaaccatcgagtctttgaacgcaagttgcgcccgaagccatcaggcctagggcacgtctgcctgggcgtcacgcatctcgtcgccgcccccacctgtcgtgtggggcggcggatgttggcctcccgtgccgttcggcgcggccggcctaaatgagagtcctcggcgagggacgtcacgacgagtggtggttgaatgcccccgactcgatttttgttgtgccggttcccgtcgtcgtattcggctccacggatgaccctagtgcgcgatctgttcgcagtcgcgccccgaca

SEQ ID NO: 44 - DNA, 608 bp, Uncaria sessilifructus (Yunnan)
  misc_feature (248)..(284): n is a, c, g, or t
tcgaatcctgcgaaacgcacgaccgcgaacctgtgtgaacaatcgggcgtcgggtggtaggggagactaagccctccactcccccccggcgctccccgcgcgctcgtcgcgcggaaaacgtaactcaaacccggcgcggaacgcgccaaggaaaactcaataggactgccgagccctcgatgccccgtacgcggtgtgctcggggtgctgtggcgcctgtcgtaatccaaacgactctcggcaacggnnnnnnnnnnnnnnnnnnnnnnnnnnnnnnnnnnnnnatgcgatacttggtgtgaattgcagaatcccgtgaaccatcgagtctttgaacgcaagttgcgcccgaagccatcaggcctagggcacgtctgcctgggcgtcacgcatctcgtcgccgcccccacctgtcgtgtggggcggcggatgttggcctcccgtgccgttcggcgcggccggcctaaatgagagtcctcggcgagggacgtcacgacgagtggtggttgaatgcccccgactcgatttttgttgtgccggttcccgtcgtcgtattcggctccacggatgaccctagtgcgcgatctgttcgcagtcgcgccccgaca

SEQ ID NO: 45 - DNA, 607 bp, Uncaria laevigata (Yunnan); identical to SEQ ID NO: 9
  misc_feature (258)..(283): n is a, c, g, or t

SEQ ID NO: 46 - DNA, 607 bp, Uncaria laevigata (Laos)
tcgaatcctgcgaaacgcacgaccgcgaacctgtgtgaacaatcgggcgtcgggtggtaggggagactaagccctccgttcccgcccggcgctccccgcgcgctcgtcgcgcggaaaacgtaactcaaacccggcgcggaacgcgccaaggaaaactcaataggactgccgagccctcgatgccccgtacgcggtgtgctcggggtgctgtggcgcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggcctagggcacgtctgcctgggcgtcacgcatcgcgtcgccgcccccacctgtcgtgtggggcggcggatgttggcctcccgtgccgttgggcgcggccggcctaaatgagagtcctcggcgagggacgtcacgacgagtggtggttgaatgacccgactcgagttttgttgtgccggttcccgtcgtcgtcttcggctccacggatgaccctagtgcgcgatctgttcgcagtcgcgccccgacc

SEQ ID NO: 47 - DNA, 606 bp, Uncaria macrophylla (Yunnan)
tcgaatcctgcgaaacgcacgaccgcgaacctgtgtgaacaaccgggcgtcgggtggtaggggagactaagccctccattcccacccggcgctccccgcgcgctcgtcgcgcggaaaacgtaactcaaacccggcgcggaacgcgccaaggaaaactcaataggactgtcgggcccccgatgccccgtacgcggtgtgctcggggtgccgcggcgcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggccagggcacgtctgcctgggcgtcacgcatcgcgtcgccgcccccacccstcgtgtggggcggcggatgttggcctcccgtgccgttaggcgcggccggcctaaatgagagtcctcggcgagggacgtcacgacgagtggtggttgaatgccccgactcgagttttgtcgtgccggttcccatcgtcgtcttcggctccacggatgaccctagtgcgcgatctgtttgcagtcgcgccccgacc

SEQ ID NO: 48 - DNA, 606 bp, Uncaria macrophylla (Yunnan)
  misc_feature (236)..(286): n is a, c, g, or t
tcgaatcctgcgaaacgcacgaccgcgaacctgtgtgaacaaccgggcgtcgggtggtaggggagactaagccctccattcccacccggcgctccccgcgcgctcgtcgcgcggaaaacgtaactcaamcccggcgcggaacgcgccaaggaaaactcaataggactgtcgggcccccgatgccccgtacgcggtgtgctcggggtgccgcggcgcctgtcgtaatccaaacgacnnnnnnnnnnnnnnnnnnnnnnnnnnnnnnnnnnnnnnnnnnnnnnnnnnngcgatacttggtgtgaattgcagaatcccgtgaaccatcgagtctttgaacgcaagttgcgcccgaagccatcaggccagggcacgtctgcctgggcgtcacgcatcgcgtcgccgcccccacccgtcgtgtggggcggcggatgttggcctcccgtgccgttaggcgcggccggcctaaatgagagtcctcggcgagggacgtcacgacgagtggtggttgaatgccccgactcgagttttgtcgtgccggttcccatcgtcgtcttcggctccacggatgaccctagtgcgcgatctgtttgcagtcgcgccccgacc

SEQ ID NO: 49 - DNA, 606 bp, Uncaria macrophylla (Yunnan)
tcgaatcctgcgaaacgcacgaccgcgaacctgtgtgaacraccgggcgtcgggtggtaggggagactaagccctccattcccacccggcgctccccgcgcgctcgtcgcgcggaaaacgtaactcaaacccggcgcggaacgcgccaaggaaaactcaataggactgtcgggcccccgatgccccgtacgcggtgtgctcggggtgccgcggcgcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggccagggcacgtctgcctgggcgtcacgcatcgcgtcgccgcccccacccgtcgtgtggggcggcggatgttggcctcccgtgccgttaggcgcggccggcctaaatgagagtcctcggcgagggacgtcacgacgagtggtggttgaatgccccgactcgagttttgtcgtgccggttcccatcgtcgtcttcggctccacggatgaccctagtgcgcgatctgtttgcagtcgcgccccgacc

SEQ ID NO: 50 - DNA, 606 bp, Uncaria macrophylla (Guangxi)
tcgaatcctgcgaaacgcacgaccgcgaacctgtgtgaacaaccgggcgtcgggtggtaggggagactaagccctccattcccacccggcgctccccgcgcgctcgtcgcgcggaaaacgtaactcaaacccggcgcggaacgcgccaaggaaaactcaataggactgtcgggcccccgatgccccgtacgcggtgtgctcggggtgccgcggcgcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggccagggcacgtctgcctgggcgtcacgcatcgcgtcgccgcccccacccgtcgtgtggggcggcggatgttggcctcccgtgccgttaggcgcggccggcctaaatgagagtcctcggcgagggacgtcacgacgagtggtggttgaatgccccgactcgagttttgtcgtgccggttcccatcgtcgtcttcggctccacggatgaccctagtgcgcgatctgtttgcagtcgcgccccgacc

SEQ ID NO: 51 - DNA, 606 bp, Uncaria macrophylla (Laos)
  misc_feature (260)..(283): n is a, c, g, or t
tcgaatcctgcgaaacgcacgaccgcgaacctgtgtgaacaaccgggcgtcgggtggtaggggagactaagccctccattcccacccggcgctccccgcgcgctcgtcgcgcggaaaacgtaactcaaacccggcgcggaacgcgccaaggaaaactcaataggactgtcgggcccccgatgccccgtacgcggtgtgctcggggtgccgcggcgcctgtcgtaatccaaacgactctcggcaacggatatctcggctcnnnnnnnnnnnnnnnnnnnnnnnnaatgcgatacttggtgtgaattgcagaatcccgtgaaccatcgagtctttgaacgcaagttgcgcccgaagccatcaggccagggcacgtctgcctgggcgtcacgcatcgcgtcgccgcccccacccstcgtgtggggcggcggatgttggcctcccgtgccgttaggcgcggccggcctaaatgagagtcctcggcgagggacgtcacgacgagtggtggttgaatgccccgactcgagttttgtcgtgccggttcccatcgtcgtcttcggctccacggatgaccctagtgcgcgatctgtttgcagtcgcgccccgacc

SEQ ID NO: 52 - DNA, 607 bp, Uncaria yunnanensis (Yunnan)
tcgaatcctgcaaaacgcacgaccgcgaacctgtgtgaacaatcgggcgtcgggtggtaggggagactaagccctccattcccacccggcgctccccgcgcgcccgtcgcgcggaaaacgtaactcaaacccggcgcggaacgcgccaaggaaaactcaataggactgtcgggccctcgatgccccgtacgcggtgtgctcggggtgccgcggcgcctgtcgtaatccaaacgactctcggcaacggatatctcggctctcgcatcgatgaagaacgtagcgaaatgcgatacttggtgtgaattgcagaatcccgtgaaccatcgagtctttgaacgcaagttgcgcccgaagccatcaggccagggcacgtctgcctgggcgtcacgcatcgcgtcgccgcccccacccgtcgtgtggggcggcggatgttggcctcccgtgccgttaggcgcggccggcctaaatgagagtcctcggcgagggacgtcacgacgagtggtggttgaatgccccgactcgagttttgttgtgccggttcccatcgtcgtcttcggctccacggattgaccctagtgcgcgatccgattgcagtcgcgccccgacc

SEQ ID NO: 53 - DNA, 607 bp, Uncaria yunnanensis (Laos); identical to SEQ ID NO: 11
  misc_feature (257)..(285): n is a, c, g, or t

SEQ ID NO: 54 - DNA, 22 bp, Artificial
  primer
ggaagtaaaagtcgtaacaagg

SEQ ID NO: 55 - DNA, 20 bp, Artificial
  primer
tcctccgcttattgatatgc

SEQ ID NO: 56 - DNA, 20 bp, Artificial
  primer
gctgcgttcttcatcgatgc

SEQ ID NO: 57 - DNA, 20 bp, Artificial
  primer
gcatcgatgaagaacgcagc

SEQ ID NO: 58 - DNA, 22 bp, Artificial
  primer
gagttttccttggcgcgttccg

SEQ ID NO: 59 - DNA, 21 bp, Artificial
  primer
cgcgcggaaaacgtaactcaa

SEQ ID NO: 60 - DNA, 21 bp, Artificial
  primer
attcaaccaccactcgtcgtg

SEQ ID NO: 61 - DNA, 20 bp, Artificial
  primer
ctaaatgagagtcctcggcg